(19) United States
(12) Patent Application Publication
(10) Pub. No.: US 2009/0297493 A1
(43) Pub. Date: Dec. 3, 2009

(54) NANOPOROUS PARTICLE WITH A RETAINED TARGET

(76) Inventor: David Anderson, Colonial Heights, VA (US)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 12/541,737

(22) Filed: Aug. 14, 2009

Related U.S. Application Data
(63) Continuation-in-part of application No. 10/170,214, filed on Jun. 13, 2002, now abandoned.

(51) Int. Cl. G01N 33/53; C12Q 1/68; A61K 38/43; A61P 35/00

(52) U.S. Cl. 424/94.1; 435/7.1; 435/6

(57) ABSTRACT

Porous nanostructured materials, such as porous nanostructured liquid and liquid crystalline particles or materials, incorporate a target substantially within the material which selectively binds a chemical of interest, which can diffusion within the porous nanostructured material and be bound by the target. The porous nanostructured materials can be dispersed as particles in a medium in which said chemical of interest is located with low turbidity. Markers which detect binding of said chemical of interest can be maintained in the medium separate and apart from the target, and any active compound (e.g., an enzyme) associated therewith by the porous nanostructured material, such that detectable changes in the marker only result when the active compounds diffuse out of the porous nanostructured materials after the chemical of interest binds to the target.

### BACKGROUND OF THE INVENTION

1. Field of the Invention

The present invention generally relates to specific interactions for binding chemicals or analytes of interest. In particular, the invention pertains to diagnostic assays which operate by specific target-analyte binding interactions, as well as to separation or segregation devices where specified chemical compounds can be sequestered from a fluid medium inside a particle or material dispersed within the fluid medium using a specific binding interaction. The present invention also pertains to selective delivery of chemicals in vitro. ex vivo and in vivo.

2. Background Description

Current assay methodologies. e.g., for the analysis of blood and other body fluids in clinical settings or for pharmaceutical screening, can be divided into two general types. Solid-phase assays require a (bio)chemically active surface, usually of a plastic plate or “well”, over which a sample of liquid (often blood or urine) is added, at which point a reaction or more typically a binding displacement occurs at this solid surface as a central step in the analysis. Typically the solid surface will be the site of an attached biomolecule, such as an antibody, receptor, ligand, nucleic acid, oligosaccharide, or other compound to which an analyte can bind. Liquid phase assays, on the other hand, are performed in a solution or dispersion without need for an active solid surface. Since solid-phase assays generally require special equipment such as plate readers, and involve higher materials costs and in some cases labor-intensive steps such as multiple plate rinses, they are usually much more. expensive than liquid-phase assays particularly from a clinical perspective.

The availability of a widely-applicable system that would move current solid-phase assays into the liquid-phase domain could make the purchase of specialized solid-phase equipment obsolete, resulting in savings to the health-care industry in both equipment and personnel costs, and provide for faster response time in the analysis of critical body fluids and in high-throughput pharmaceutical screening.

The development and broad applicability of solid-phase assays, e.g., to such areas as pharmaceutical drug design and screening, is hindered by the strong tendency of solid surfaces—such as the polystyrene plates that are widely used as substrata—to denature the molecules that are purposefully bound to the surface. Such denaturation can adversely affect the ability of the bound molecule to exhibit the proper conformation and conformation-dependent binding selectivity.

Development of accurate assays employing biomacromolecules, particularly receptor proteins, is of central importance in biomedical and pharmaceutical advancement. For example, research into the actions of pharmacologic agents can be illuminated in many cases by biochemical dissection of the effects of drugs on receptor mechanisms. In particular, there is abundant evidence for differences between species, and even between members of the same species, in mechanisms and intensity of hormone and receptor regulation. Such regulatory feedback has the capacity to alter responses, which are further contingent on interactions between different hormonal or neutotransmitter systems. It is therefore important to interpret pharmacologic endpoint results in the context of biochemical data concerning direct receptor effects of drugs.

However, existing methods for the immobilization of membrane proteins, receptors in particular, for systematic studies are highly problematical and inherently limited. There is no question about the need for a lipid-based matrix, not only for proper function (activity) of the receptor but even for the proper presentation of the protein in studies of ligand, drug, or antibody binding. This has been amply demonstrated for proteins such as the acetylcholine receptor (AChR), LHRH receptor, insulin receptor, TSH receptor, and EGF receptor, where allosteric regulation of binding affinity is a central issue. Proper protein presentation becomes even more critical for drug screening employing multisite receptors such as AChR and κ-opioid receptor, where the endogenous ligands and exogenous agonists/antagonists can bind to different sites.

Non-lipidic, nanoporous matrices offer high surface areas but no lipid bilayer, and are generally not available in microparticle dispersion format. Furthermore, the pore size distribution in most nanoporous materials is either too broad, or centered on an average value which is too large, to allow size-based exclusion of the level of sophistication utilized in the present invention.

For receptor-based assays, allosteric effects involving the global protein, which drive signal transduction, are in many proteins driven by the lower free energy associated with binding site/ligand interaction—and thus tighter binding—after conformational change. In other words, the original binding configuration leaves room for improvement, so that the driving force for a tighter fit induces the conformational change. Thus, in the absence of the entire protein and associated allosteric effects, as for example when soluble fragments of receptor proteins are employed rather than intact proteins, studies of competitive binding can be qualitatively incorrect. For instance, while binding sites of nAChR are in the α-subunits, their affinity and cooperativity are regulated by the γ- and δ-subunits. In certain multisite receptors such as AChR and κ-opioid receptor, it is known that the natural ligand and exogenous agonists/antagonists can bind to different sites. Thus, a pharmaceutical assay based on a partially expressed protein exhibiting only the natural ligand binding site would yield false negatives with exogenous compounds, and the opportunity afforded by the new potential drug might well be missed. Furthermore in receptors for biogenic amine neurotransmitters, such as the 5-HT-2c receptor (where the binding site involves a transmembrane domain) as well as in cases where the site is at the membrane/water interface or (as in AChR) at the interface between two subunits, it would be erroneous to work only with a partially expressed protein representing a putative binding site. Discrimination between agonist and antagonist binding sites will clearly require intact receptor, as in the case of human thromboxane A2 receptor, where a single conservative amino acid substitution in the seventh hydrophobic transmembrane helix has been shown to discriminate these two sites. As another example, dimerization of the EGF receptor, which has a strong effect on binding affinity, apparently requires intact receptors and receptor-related molecules such as the secreted binding domain and gp74v-erbB do not give evidence of dimerization. The need for a receptor with its allosteric regulatory mechanism intact, and with proper presentation and accessibility of binding site(s), then implies the need for a bilayer milieu. In addition, the role of lipid-protein and lipid-peptide interactions is of direct impact on binding events. Recent data on the nAChR, for example, indicate that interactions with the membrane bilayer at the so-called “lipid-protein interface” determine tertiary structure and receptor conformation, which is critical in binding affinity. In addition, the effect of lipid interactions in conferring proper structure to peptide hormone ligands that are unstructured in water is well established.

For applications of membrane proteins in pharmaceutical screening, biosensors, immunoassays, affinity separations, bioreactors, etc., none of the currently available protein matrices meet all the desirable properties for a system incorporating a membrane-associated protein: i) a purified system free of extraneous proteins that complicate analysis; ii) a lipid bilayer milieu, iii) a large surface area of bilayer facilitating high protein loadings for sensitivity; iv) long-term stability; and v) capable of being dispersed in submicron form so as to yield dispersions of low turbidity suitable for optical or spectroscopic analyses. Previous artificial (or “biomimetic”) bilayer systems that have been useful in the study of membrane proteins—but far less useful in their technological applicator—are liposomes, BLMs, and L-B films. None of these satisfy all the above criteria. Liposome-based systems suffer from instability, low loadings, non-reproducibility, protein orientation/accessibility problems. The use of cell fragments inevitably suffers from unavoidable contaminants and components that complicate interpretation of results. Lipidated beads. which arc polymer beads coated with a monolayer of lipid, are clearly not suitable for transmembrane proteins. Furthermore, in matrices such as these, the protein is necessarily bound at the surface of the particle, introducing the limitations discussed above. Enzyyme electrode biosensors are based on a Langmuir-Blodgett film of lipid deposited on a metal electrode, and binding is detected by conductance changes due to adsorption; in the case of receptors, these cannot measure activity, are interfered with by non-specific binding, can denature sensitive proteins, have very low loadings, and often show marginal signal at best in the case of a low-MW ligand (neurotransmitter) binding to a high-MW receptor protein on a 1:1 molar basis. Other systems that have served as matrices for the immobilization of proteins, but are not lipid-based, are nanoporous materials such as controlled-pore glass, agarose and other gels, track--etch membranes, and other high-surface area materials. With these matrices, attachment of the protein is accomplished by covalent bonding or adsorption. either of which are problematic for maintaining conformation.

U.S. Patent 6,256.522 and, Schultz, J. S, Mansouri, S. and Goldstein, I. J. “Affinity sensor. a new technique for developing implantable sensors for glucose and other metabolites.” Diabetes Care, 1982, 5: 245, describe affinity sensors for monitoring various metabolites in blood plasma by optical means. In these references, the principle of detection is similar to that used in radioimmnunoassays and is based on the competitive binding of a particular metabolite and a fluorescein-labeled analogue with receptor sites specific for the metabolite and the labeled ligand. The references describe an affinity sensor for glucose. Concanavalin A, a protein with specific binding character for glucose, was immobilized on the inside surface of a hollow dialysis fiber. Fluorescein-labeled dextran was selected as the competitive labeled ligand. The molecular weight cutoff of the dialysis fiber is low enough Lo completely retain the 70,000 MW dextran within the fiber lumen while glucose can freely pass through the dialysis membrane. The sensor is completed by inserting a single optical fiber in the lumen of the dialysis fiber, thus allowing measurement of the unbound FITC-dextran.

Ballerstadt, R.; Schultz, J. S., Anal Chem VOL., 72 NO. 17 2000 Sep 1 PP. 4185-92 describes a fluorescence affinity hollow fiber sensor for transdermal glucose monitoring The glucose-sensing principle is based on the competitive reversible binding of a mobile fluorophore-labeled Concanavalin A (Con A) to immobile pendant glucose moitles inside of intensely colored Sephadex beads. The highly porous beads (molecular weight cutoff of 200 kDa) were colored with two red dyes, Safranin O and Pararosanilin, selected to block the excitation and spectrum of the fluorophore Alexa488. The sensor consists of the dyed heads and Alexa488-Con A confined inside a scaled, small segment of a hollow fiber dialysis membrane (diameter 0.5 mm, length 0.5 cm. molecular weight cutoff 10 kDa). In the absence of glucose, the majority of Alexa488-Con A resides inside the colored beads bound to fixed glucose. Excitation light at 490 nm impinging on the sensor is strongly absorbed by the dyes, resulting in a drastically reduced fluorescence emission at 520 nm from the Alexa488-Con A residing within the beads. However, when the hollow fiber sensor is exposed to glucose, glucose diffuses through the membrane into the sensor chamber and competitively displaces Alexa 488-Con A molecules from the glucose residues of the Sephadex beads. Thus, Alexa 488-Con A appears in the void space outside of the beads and is fully exposed to the excitation light, and a strong increase in fluorescence emission at 520 nm is measured.

U.S. Patent Application 20020206346 to Engstrom describes the use of a liquid crystalline phase for determination of the distribution of a chemical substance between a hydrophobic and a hydrophilic phase.

### SUMMARY OF THE INVENTION

It is an object of the invention to provide a separation or segregation device made of a porous nanostructured material, preferably cubic phase, which includes a target positioned therein. preferably at least 90% partitioned into the porous nanostructured material, which can be used in chemical separations, assays, therapeutic drug delivery, and in other applications. Preferably the nanostructured material is lyotropic meaning that it is comprised of a liquid phase or liquid crystalline phase material that contains a solvent. However, thermotropic materials, which do not include a solvent, might also be employed in the practice of this invention. For example, anhydrous strontium soaps, and soaps with other divalent counterions, over certain temperature ranges, are known to form cubic phases which are bicontinuous in the sense that the polar groups aid counterions form the continuous polar domains, while the alkane chains form continuous apolar domains. Similarly, block copolymers (e.g., polystyrene-b-polysioprene star diblock copolymers) are known to form bicontinuous cubic phases with the same morphologies as found in lipid-water systems.

It is an object of the present invention to provide a widely-applicable system for producing low-turbidity, liquid dispersions in which binding molecules, including but not limited to antibodies and membrane-associated proteins, as well as complexes between such binding molecules and conjugated ligands, can be incorporated in such a manner that they are accessible to small molecule analytes but not to sufficiently large macromolecules.

It is a further object of the invention to provide for liquid-based assay systems in cases where solid-phase assays must currently be utilized It is a further object of the invention to provide for convenient, purified, stable, arid sensitive assay systems incorporating receptor proteins or other bilayer-associated molecules.

According to the invention, a target compound capable of binding a chemical of interest is partitioned into a porous nanostructured material. preferably a nanostructured liquid or liquid crystalline particle or material selected from the group consisting of reversed bicontinuous cubic phase. reversed hexagonal phase, L3 phase, normal bicontinous cubic phase, and normal hexagonal phase. In a competitive assay or simple segregation application of the invention, the chemical of interest will diffuse into the porous nanostructured liquid or liquid crystalline particle or material and bind to the target. hi competitive assays, a displaceable chemical such as an enzyme group or the like will be displaced by the chemical of interest and will diffuse out of die porous nanostructured liquid or liquid crystalline particle and react with a marker compound to indicate binding has occurred within the particle or material. Thus, the nanostructured liquid or liquid crystalline particle keeps the enzyme or other displaceable groups separate from the marker compound until it is released from the target, thereby allowing accurate detection without complex washing, aspiration and other processes used in many of today's automated immununoassay analyzers. This allows clinicians to conduct tests quickly and accurately, without sophisticated training or instrumentation. In a sandwich assay application of the invention, a ligand is bound to the target within the porous nanostructured liquid or liquid crystalline particle or material, or can become bound lo the target by diffusion through the porous nanostructured liquid or liquid crystalline material. hi addition, a second target which can diffuse through the nanostructured liquid or liquid crystalline material is added which binds to another epitope of the ligand. Once the second target is hound, an indication is provided demonstrating the binding.

Alternative uses of the invention are in chemical isolation and clean up, or in the delivery of drugs, enzymes, or other bioactive agent, e.g., radioactive agents and chemical toxins. In the chemical isolation application, the particles or materials of the present invention arc simply brought into contact with a medium in which segregation and isolation or a chemical of interest is desired. Over a period of time, and with or without operations such as stirring, agitation, etc., the chemical diffuses within the porous nanostructured liquid or liquid crystalline particle or material and is bound by the target. This process may be used in the clean up of contaminated water, or in the ex vivo clean up of blood, for example. In the delivery mode, the porous nanostructured liquid or liquid crystalline particle or material would incorporate a chemical to be delivered (e.g., an agonist, antagonist, medicament, toxin, etc.). This chemical would he protected from the environment, e.g. the body in an ill vivo application, by the porous nanostructured liquid or liquid crystalline particle or material, until it is in position for delivery of the chemical. Once in position, a compound from the environment will diffuse through the porous nanostructured liquid or liquid crystalline particle or material, competitively interact with the target and displace the chemical to be delivered, and, thereafter. the chemical to be delivered will diffuse out of the porous nanostructured liquid or liquid crystalline particle or material and into the environment in which it should act. In one example, an agonist of serotonin might be delivered when an antagonist is taken up from the body. Ii another example, a chemical or radioactive toxin might be delivered at a tumor or cancerous tissue cite in response to a chemical being produced by the tumor or cancerous tissue or in response to other environmental factors.

### BRIEF DESCRIPTION OF THE DRAWINGS

The foregoing and other objects aspects and advantages will be better understood from the following detailed description of a preferred embodiment of the invention with reference to the drawings, in which:

FIG. 1 is a schematic diagram illustrating one embodiment of the invention wherein an analyte in a medium diffuses into a nanoporous, nanostructured lyotropic particle or material having a target retained therein, and where competitive displacement causes release of an enzyme packet from the lyotropic particle or material which then diffuses to the medium and interacts with markers bound to a substrate, polymer or the like which is to(.) large to diffuse within the lyotropic particle or material: and

FIG. 2 is a schematic diagram illustrating a dispersion of particles according to the instant invention.

### DETAILED DESCRIPTION OF A PREFERRED

### Embodiment of the Invention

While certain protein substrate materials can be dispersed in water in microparticulate form, there has been a fundamental unsolved challenge to the design of a liquid-phase assay system based on competitive binding, to, e.g., an antibody or receptor protein. Specifically. the displacement of a marker molecule from a binding site of a protein, by the presence of the analyte to he detected, must allow the marker to diffuse somewhere it can be detected, somewhere of course the bound marker cannot easily access. As a general example, if changes in a marker are lo be detected by virtue of a chemical group that triggers the activity of a specific enzyme, for example by an incorporated nitrophenylphosphate group that is cleaved by alkaline phosphatase, then it is required that the bound marker be inaccessible to the enzyme but accessible to it after displacement. In this context, it is important to note that in nearly every marker conjugate currently available, the chemical group which activates the enzyme or other detection scheme is distinct from the epitope that binds to the bound protein, meaning that the enzyme-activating group is accessible whenever the marker is at the outer surface of a particle, whether it is bound or not. This presumably rules out any scheme in which an antibody or receptor is attached predominantly to the outer surface of a carrier particle.

Thus, this invention is directed to a segregating device for separation, analysis, etc. where a significant fraction, e.g., 90)% of the target which binds the chemical or analyte of interest, is sterically isolated from components of a medium. The segregating or separation device is preferably a porous nanostructured liquid or liquid crystalline particle or material, and most preferably a lyotropic material, and the target is maintained in the isolated state by partitioning the target into the porous nanostructured liquid or liquid crystalline material or particle. Other nanoporous materials might also be used in the context of this invention, e.g., ceramics (alumina or silica), etc. In the context of this invention a lyotropic material should he understood to be a material that is a nanostructured liquid or liquid crystalline phase selected from reversed bicontinuous cubic phase, reversed hexagonal phase, L3 phase. normal hexagonal phase. or normal bicontinuous phase, and which includes solvent within the particle or material (usually water); a lyotropic particle is similarly defined to be a particle comprising one or more of these phases. These nanostructured liquid or liquid crystalline particles or materials are deemed to be porous. and more specifically “nanoporous”, in that they exhibit a system of solvent filled (usually water filled) pores whose diameter falls within the range of 1-100 nm.

In some circumstances within the practice of this invention, it can be advantageous to use, as the interior matrix. a composition that yields a nanostructured liquid or liquid crystalline phase upon contact with water (or more rarely, other polar solvent), whether or not this dehydrated composition itself is a nanostructured liquid or liquid crystalline phase. In particular, this contact with water or a water-containing mixture could be either during a reconstitution step, or more preferably, during the application of the particle to an aqueous solution such as blood, extracellular fluid, intracellular fluid, mucuos, intestinal fluid, etc. There are several reasons why such dehydrated variants of the nanostructured liquid or liquid crystalline particles of this invention may be advantageous: to protect hydrolytically unstable actives or excipients; to limit premature release of water soluble actives; and as a natural result of a production process such as spray-drying or freeze-drying that can induce dehydration. Removal of most, or all. of the water from a nanostructured liquid or liquid crystalline phase will often yield another nanostructured liquid or liquid crystalline phase, but can sometimes yield a structureless solution, precipitate, or a mixture of these with one or more nanostructured liquid or liquid crystalline phases. In any case, for many applications, it is the hydrated form that is important in the application of the particles, and thus, if this hydrated form is a nanostructured liquid or liquid crystalline phase, then it should be understood by those of skill in the art that the composition of matter falls within the scope of the current invention.

The segregating or separation devices can take any of the following forms: 1) a nanoporous material bound to a support or substrate; 2) a nanoporous material dispersed in a medium as particles; or 3) a solid material embedded with or within a nanoporous material or nanoporous particle.

The chemical or analyte to be sequestered by the segregating or separating device is of a size and chemical constitution which permits its diffusion into tie lyotropic material to interact with and bind to the target. Binding can be detected using enzyme/marker combinations or by any other means where an enzyme or other reactive compound is used to generate a detectable change (e.g., changes in absorbance, color, turbidity, fluorescence, phosphorescence, chemiluminescence, etc.). The invention is applicable to competitive and sandwich assays. Furthermore, the ability to maintain marker and enzyme separate from each other until an assay is run, due to the presence of the target within the lyotropic material or particle and not generally exposed to the media, permits the assays to be performed without special operations such as washing, fluid aspiration, and the like.

In addition, the invention may also be used for the selective delivery of compounds, e.g., enzymes, agonists, antagonists, etc., wherein the delivered compound is released from the target only upon a specific operation. Because the delivered compound is maintained bound within the lyotropic material or particle to the target entity, it is not degraded by the environment, body defense mechanisms, etc., until delivery is intended.

Certain nanostructured liquid and liquid crystalline phases provide materials which are simultaneously lipid-based and nanoporous, with lattice-ordered structure, featuring narrow pore size distributions. It has been discovered that these phase properties can be used to produce liquid-phase assay systems, and chemical separation or segregation systems in general, with a surprising combination of favorable features including wide applicability, purity, stability, convenience, and sensitivity. One embodiment of the invention contemplates an assay system comprising a dispersion of microparticles containing at least one nanoporous liquid or liquid crystalline phase into which is entrapped a complex A-B, wherein A is a displaceable compound that binds selectively to a target compound B which is usually, though not always, a macromolecule—e.g., an antibody, receptor, chimera, lectin, nucleic acid sequence or fragment or simulant or derivative thereof—that can bind compound A and the analyte of interest X in a competitive fashion, and outside the microparticles is present a device or compound that responds to the presence of A in a measurable fashion but is substantially excluded from the interior of the microparticles. Within the context of this invention, a fragment is understood to be a portion of a target binding compound which retains the binding capability of the target binding compound, and a simulant or derivative should be understood to be any compound that is deliberately produced or selected for its ability to bind, with a selectivity that is consistent with the desired functionality of the system. the analyte or chemical of interest. In such a system, upon addition of the analyte X to the dispersion, an analyte molecule can diffuse into the interior of a microparticle and displace, by competitively binding Lo B, a molecule of the compound A, which can then exit the particle and cause a response; compound A will in general have either enzymatic or reactive/catalytic activity, or contain a substrate for an enzyme or reactant that is substantially outside the particles. In the realm of small molecules., a number of compounds that fall into the classes of small peptides, functionalized lipids and surfactants (particularly charged lipids), chelating agents. small oligosaccharides (such as those that determine blood group), crown ethers, cyclodextrans, small oligonucleotides, etc. are potentially useful as targets.

In a preferred embodiment of the invention. B is an antibody to an analyte of interest X, and the molecule A is a derivative of X that also contains an enzyme-reactive group, such as a nitrophenylphosphate group. The antibody-marker complex is entrapped in a nanoporous phase such as a reversed bicontinuous cubic phase, which is dispersed in microparticulate form. and the aqueous exterior please of the dispersion contains an enzyme such as alkaline phosphatase that reacts with the marker molecule, and that is substantially excluded from the interior of the microparticles by size exclusion, possibly together with other repulsive forces of steric or ionic origin. In the absence of analyte, substantially all of the molecule A is bound in the interior of the particles by the antibody, so the signal is very low. Addition of analyte X to the dispersion induces displacement of some of the molecule A from the antibody into the exterior phase, resulting in a signal increase as the molecule A eventually contacts the enzyme. Alternatively, the molecule or enzyme packet A contains the enzymatic or catalytic/reactive moiety (typically an enzyme such as alkaline phosphatase or a peroxidase, conjugated to X or a simulant thereof), and a marker that responds to this enzymatic activity is substantially confined outside the particles, usually by size exclusion from the pores; addition of analyte X to the dispersion induces displacement of some of the molecule or enzyme packet A from the target into the exterior phase, resulting in a signal increase as A eventually contacts the marker. This latter arrangement has the advantage that a single analyte molecule can release an enzyme that can catalyze a large number of repetitive chemical reactions on the marker, giving an amplification of the signal.

In another embodiment of the invention, which is well-suited for pharmaceutical development. B is a receptor protein which is a pharmaceutical target for a particular disease or condition, and a candidate drug X is being tested for binding to B. After adding a known amount of candidate drug X, the amount of ligand A released from the nanoporous material due to displacement is determined, and the degree of binding of the drug X to receptor B is calculated from the result. In deed, from an analysis of the competition over a range of concentrations of X, the binding constant between X and the receptor can be estimated. This embodiment is particularly suited to high throughput screening such as might occur where the lyotropic material or particle of this invention is positioned in a multi-well tray or series of tubes which are tested against various drug candidates simultaneously.

The lyotropic materials useful in the practice of this invention include liquid and liquid crystalline phases or, as described above, dehydrated variants thereof, and the invention and its applications are described in conjunction with the following terms:

The nanostructure phases of utility. The phases of potential utility in chemical separation, diagnostic assays, therapeutic delivery vehicles, or other applications of the invention arc the reversed bicontinuous cubic phase. the reversed hexagonal phase. the L3 phase, and to a lesser extent the normal bicontinuous cubic and normal hexagonal phases. The most preferred is the reversed bicontinuous cubic phase. All of these phases are nanostructured phases, meaning essentially that they exhibit a microdomain structure with characteristic dimensions in the range of nanometers: about 1-100 nm in effective diameter. Nanostructured should be understood in the context of this invention as referring to the building blocks of which of the material or particle, and these are on the order of nanometers (e.g., one lo hundreds of nanometers). Generally, a material that contains domains of I to 100 nm across, or layers or filaments of that thickness can be considered a nanostructured material. The nanostructured liquid phases and liquid crystalline phase materials of this invention are characterized by having nanoscale domains which are clearly distinguished from neighboring domains by large differences in local chemical composition. A domain is characterized as a spatial region which is of chemical makeup that is clearly distinguishable from that of a neighboring domain. The definition of nanostructured liquids and liquid crystals, as well as the structures, methods of identification, are known by those of skill in the art. A brief review of the appropriate nanostructured liquid phase (the L3 phase) is first given, Followed by a review of the appropriate nanostructured liquid crystalline phases.

The nanostructured liquid phases occurring in lyotropic systems used in the practice of this invention are characterized by domain structures, composed of domains of at least a first type and a second type having the following properties:

a) the chemical moieties in the first type domains are incompatible with those in the second type domains such that they do not mix under the given conditions but rather remain as separate domains; for example, the first type domains could be composed substantially of polar moieties such as water and lipid head groups, while the second type domains could be composed substantially of apolar moieties such as hydrocarbon chains; or, first type domains could be polystyrene-rich, while second type domains are polyisoprene-rich, and third type domains are polyvinylpyrrolidone-rich;

b) the atomic ordering within each domain is liquid-like rather than solid-like, i.e., it lacks lattice-ordering of the atoms; (this would be evidenced by an absence of sharp Bragg peak reflections in wide-angle x-ray diffraction);

c) the smallest dimension (e.g., thickness in the use of layers, diameter in the case of cylinder-like or sphere-like domains) of substantially all domains is in the range of nanometers (viz., from about I to about 100 nm): and d) the organization of the domains does not exhibit long-range order nor conform to any periodic lattice. This is evidenced by the absence of sharp Bragg reflections in small-angle x-ray scattering examination of the phase. Furthermore, if high viscosity and birefringence arc both lacking, this is strong evidence of a liquid, as opposed to liquid crystalline. phase.

The L3 phase. 1′he nanostructured liquid phase known as the L3 phase is also called the “sponge phase”, or “anomolous phase”, and has a bicontinuous structure related to the bicontinous cubic phase, hut lacking in long-range order. Certain L3 phases (of the bilayer type generally) arc most appropriately dispersed in (or placed in contact with) polar solvent, whereas others (of the monolayer type) are most appropriately dispersed in an apolar solvent. for the purposes of this invention.

L3 phases occur in phase diagrams as isolated islands, or as (apparent) extensions of L2 (or LI) phase regions. That is, L2-phase regions in phase diagrams sometimes exhibit ‘tongues’ sticking out of them: long, thin protrusions unlike the normal appearance of a simple L2 phase region. This sometimes appears also with some L1 regions, as described below. When one examines these closely, especially with X-ray and neutron scattering, they differ in a fundamental way from L2 phases. In an L2 phase, the surfactant film is generally in the form of a monolayer, with oil (apolar solvent ) on one side and water (polar solvent) on the other. By contrast, in this “L3 phase.” as these phases arc called, the surfactant is in the form of a bilayer, with water (polar solvent) on both sides. The L3 phase is generally considered to be bicontinuous and, ill fact. it shares another property with cubic phases: there are two distinct aqueous networks, interwoven but separated by the bilayer. So, the L3 phase is really very similar to the cubic phase. but lacking the long-range order of the cubic phase. 1,3 phases stemming from L2 phases aid those stemming from LI phases are given different names. “1w phase” is used for those associated to L2 phases, and “L3* phase” for those associated to LI phases.

In spite of its optical isotropy when acquiescent and the fact that it is a liquid, the L3 phase can have the interesting property that. it can exhibit flow birefringence. Often this is associated with fairly high viscosity, e.g., viscosity that can be considerably higher than that observed in the L1 and L2 phases, and comparable to or higher than that in the lamellar phase. These properties are of course a result of the continuous bilayer film, which places large constraints on the topology. and the geometry, of the nanostructure. Thus, shear can result in the cooperative deformation (and resulting alignment) of large portions of the bilayer film, in contrast with, for example. a micellar L1 phase, where independent micellar units can simply displace with shear, displace with shear, and in any case a monolayer is generally much more deformable under shear than a bilayer. Support for this interpretation comes from the fact that the viscosity of L3 phases is typically a linear function of the volume fraction of surfactant.

As a component of the particle the nanostructured liquid crystalline phase material may be a nanostructured reversed bicontinuous cubic phase material;

a nanostructured reversed hexagonal phase material;

a nanostructure(i normal hexagonal phase material; or a nanostructured normal bicontinuous cubic phase material.

The nanostructured liquid crystalline phases are characterized by domain structures. composed of domains of at least a first type and a second type (and in some cases three or even more types of domains) having the same properties a-c as listed above for nanostructured liquids. The organization of the domains conforms to a lattice. which may be one-. two-, or three-dimensional, and which has a lattice parameter (or unit cell size) in the nanometer range (viz., from about 5 Lo about 200 rim); the organization of domains thus conforms to one of the 230 space groups tabulated in the International Tables of Crystallography, and would be evidenced in a well-designed small-angle x-ray scattering (SAXS) measurement by the presence of sharp Bragg reflections with d-spacings of the lowest order reflections being in the range of 3-200 nm.

The reversed bicontinous cubic phase. Such a phase has cubic crystallographic symmetry, which makes it optically isotropic and yields characteristic indexings of the Bragg peaks in SAXS, corresponding usually to one of the space groups Im3m. Pn3m, or Ia3d. The bicontinuous property, in which both polar and apolar components are simultaneously continuous in all three dimensions, gives rise Lo high self-diffusion coefficients of all components of low MW, whether they are segregated into the polar or the apolar domains, and also gives rise to high viscosities, often in the millions of centipoise. This phase generally appears at lower water contents than lamellar phases, and/or at higher water contents than reversed hexagonal phases. and can also sometimes be induced by adding a hydrophobic component to a lamellar phases or a non-surfactant amphiphile with a weak polar group. When this is the phase used in the practice of this invention and it is desired to have this dispersed in. or in contact with, a solvent then the solvent should preferably be a polar one, typically water or aqueous buffer. but more generally a polar solvent or mixture thereof. The pore size can be adjusted by changing the composition, and be determined precisely.

The reversed hexagonal phase. The reversed hexagonal phase structure consists of long cylindrical reversed (water-core) micelles packed onto a hexagonal lattice, which can be readily confirmed by SAXS. Usually the viscosity of the reversed hexagonal phase is quite high. higher than a typical normal hexagonal phase, and approaching that of a reversed cubic phase. In terms of phase behavior, the reversed hexagonal phase generally occurs at high surfactant concentrations in double-tailed surfactant/water systems, often extending to, or close to, 100% surfactant. Usually the reversed hexagonal phase region is adjacent to the lamellar phase region which occurs at lower surfactant concentration, although bicontinuous reversed cubic phases often occur in between.

When the reversed hexagonal phase is the phase used in the practice of this invention and it is desired to have this dispersed in, or in contact with. a solvent then the solvent should preferably be a polar one, typically water or aqueous buffer, but more generally a polar solvent or mixture thereof.

The normal bicontinous cubic phase. Such a phase has a structure in which both polar (e.g., water) and apolar (e.g., surfactant chains, added oil) domains form continuous. sample-spanning paths in all three dimensions. and small-angle x-ray shows peaks indexing to a three-dimensional space group with a cubic aspect. To the unaided eye, the phase is generally transparent when fully equilibrated, and thus often considerably clearer than any nearby lamellar phase. In the polarizing optical microscope, the phase is non-birefringent, and therefore there are no optical textures. For normal bicontinuous cubic phases in surfactant-water systems, the viscosity is usually high, typically in the millions of centipoises, and no splitting is observed in the NMR bandshape, only a single peak corresponding to isotropic motion. In terms of phase behavior. the normal bicontinuous cubic phase generally occurs at fairly high surfactant concentrations in single-tailed surfactant/water systems, typically on the order of 70% surfactant with ionic surfactants. Usually the normal bicontinuous cubic phase region is between lamellar and normal hexagonal phase regions, which along with its high viscosity aid non-birefringence make its determination fairly simple. In double-tailed surfactants, it generally does not occur at all ill the binary surfactant-water system.

When the normal bicontinous cubic phase is the phase used in the practice of this invention and it is desired to have this dispersed in, or in contact with, a solvent then the solvent should preferably be an apolar one.

The normal hexagonal phase. The normal hexagonal phase structure comprises long cylindrical micelles packed onto a hexagonal lattice, which can be readily confirmed by SAXS. Usually the viscosity is moderate, more viscous than the lamellar phase but far less viscous than typical cubic phases (which have viscosities in the millions of centipoise). The self-diffusion coefficient of the surfactant is slow compared to that in the lamellar phase; than of water is comparable to that in bulk water. The ²H NMR bandshape using deuterated surfactant shows a splitting, which is one-half the splitting observed for the lamellar phase. And in terms of phase behavior, the normal hexagonal phase generally occurs at moderate surfactant concentrations in single-tailed surfactant I water systems, typically on the order of 50% surfactant. Usually the normal hexagonal phase region is adjacent to the micellar (L1) phase region. although non-bicontinuous cubic phases can sometimes occur in between. In double-tailed surfactants, it generally does not occur at all in the binary surfactant-water system.

When the normal hexagonal phase is the phase used in the practice of the invention and it is desired to have this dispersed in, or in contact with, a solvent then the solvent should preferably be an apolar one.

Polar solvent: in the context of the instant invention, a polar solvent may be for example one of the following or a mixture thereof: water, glycerol. ethylene glycol, acetamide, N-methylacetamide, N,N-dimethylacetamide, formamide, N-methylformamide, N,N-dimethylformamide, N-methyl sydnone, ethylammonium nitrate, and polyethylene glycol of low MW (e.g., less than about 1,000). Other polar solvents may also be employed in the practice of this invention.

Dispersions of liquid crystalline particles. In a preferred embodiment of the invention, the target will be positioned in liquid crystalline particles as discussed above. The liquid crystalline particles may be constructed from the following types of materials: surfactants, polar lipids (phospholipids, glycolipids, sphingolipids, etc.), block copolymers (particularly amphilic bock copolymers), etc. The liquid crystalline particles will preferably have a diameter ranging from 30 to 300 run, and more preferably ranging from 50 to 200 nm, and most. preferably ranging from 50 to 150 nm. A number of methods are available for dispersing the lyotropic liquid crystalline phase particles or materials in solvents. Dispersing the porous cubic and hexagonal phases is in some respects different from dispersing the lamellar phase. The method used in the formation of lipsomes. e.g., sonicating lamellar phase or lamellar phase-forming lipids in water, often does- not work with cubic and hexagonal phases because fragments of the latter phases seem to fuse more readily with each other, apparently because of the porosity, which is related to the intrinsic curvature in the monolayers that make up the structures (see, Anderson, Wennerstrom, and Olsson: “Isotropic, bicontinuous solutions in surfactant-solvent systems: the L3 phase”, J. Phys. Chem. 1989, 93:4532-4542). Most of the work with dispersing reversed cubic and hexagonal phases has focused on dispersing these phases in water. Significantly, in U.S. patent application Ser. No. 09/297,997 filed Aug. 16, 2000 (notice of allowance issued and issue fee paid), the complete contents of which is herein incorporated by reference, the present inventor described methods for producing dispersions of coated particles of a wide range of liquid crystalline phases including cubic and hexagonal. These methods include chemical reactions, heating-cooling temperature cycles, acid-base reactions, and other methods for forming solid, typically brittle coating phases in combination with sonication or other steps for cracking the material into coated particles containing liquid crystal. Such particle-producing methodology is particularly useful in the instant invention because it provides for particles that, while still coated, can be handled as solids and/or can protect sensitive interior components (most notably proteins) as well as phase structures from change during storage. The coating can also shield the marker from enzyme until the coating is dissolved away, for example by dilution with water as in the Examples given below. U.S. Pat. 5,531,925 (Landh and Larsson) describes methods for producing particles of reversed cubic and reversed hexagonal phases with a distinct surface phase comprising a lamellar. crystalline lamellar, or L3 phase. While the techniques used in that patent are of interest in the present connection, the particles in which the surface phase is a lamellar or crystalline lamellar phase are not useful per sc in the instant invention, because the do not allow diffusion of the analyte into the liquid crystal or flow of the enzyme (or marker) out; particles with the porous liquid L3 coating on the other hand are of potential use in the instant invention since they do allow diffusion in and out.

Besides the use of distinct phases such as the L3 phase or a solid coating phase. the inventor has found that ionic stabilization and steric stabilization provide means by which to stabilize dispersions of even fusion-prone reversed cubic and hexagonal phases. If a properly-chosen ionic surfactant is combined with a nonionic surfactant and water at, a composition that is found to be a reversed cubic phase, for examplc, then provided the concentration of the ionic surfactant (which can he either anionic or cationic) is high enough that a surface charge of at least about 30 mV, and preferably greater than about 40 mV, exists at the surface of the liquid crystal, then it is generally possible to disperse the liquid crystal with the application of ordinary homogenization means-though the strongly preferred method is high-pressure microfluidization. With surfactants that have polar groups of relatively high MW, preferably polyethylene glycol (PEG) of MW greater than about 1,000 Daltons, steric stabilization can make dispersions of liquid crystal particles stable for considerable timespans.

The preferred method for stabilizing particles in the instant invention is to produce coated particles according to U.S. Ser. No. 09/297,997, wherein the coating is soluble in water, and to maintain the particle in coated form during its storage life; then upon use, the addition of water can dissolve the coat and at the same time re-disperse the (now uncoated) microparticles. stabilized now by steric stabilization, ionic stabilization, or the presence of an L3 surface phase. In that case the shelf-life of the coated particles (which can be stored in either wet or dry format) should preferably be at least one year, more preferably 2 years, whereas the stability of the dispersion created by addition of water (or more likely buffer, or in some cases the fluid to be analyzed) need be only minutes or hours.

The normal phases above, namely the normal bicontinuous cubic and nornal hexagonal phases, can be dispersed in certain oily (hydrophobic) solvents. Such dispersions can be useful in the case where the analyte is of very low water solubility, or the solvent in the solution to be analyzed is hydrophobic (water-immiscible). Such a dispersion could be advantageous in the case where the analyte is water-soluble, but. also soluble in a more hydrophobic solvent that would exclude other confounding factors. A number of workers such as Klibanov have shown that many enzymes retain their activity in hydrophobic solvents. While normal cubic and hexagonal phases do not contain bilayers in the true sense, they nonetheless contain monolayers and even apposed monolayers that mimic a bilayer sufficiently in many cases that, the bilayer-based protein matrices discussed herein can be mimicked in these phases.

Polar: polar compounds (such as water) and polar moieties (such as the charged head groups on ionic surfactants or on lipids) arc water-loving, or hydrophilic; “polar” and “hydrophilic” in the context of the present invention are essentially synonymous. In terms of solvents, water is not the only polar solvent. Others of importance in the context of the present invention are: glycerol, ethylene glycol, formamide, N-methyl formamide, dimethylformamide, ethylammonium nitrate, and polyethylene glycol. More generally, in terms of polar groups in hydrophilic and amphiphilic molecules (including but not limited to polar solvents and surfactants). a number of polar groups are tabulated below, in the discussion of which polar groups are operative as surfactant head groups and which are not.

Apolar: Apolar (or hydrophobic, or alternatively “lipophilic”) compounds and moieties include not only the paraffinic I hydrocarbon I alkane chains of surfactants, but also modifications of them, such as perfluorinated alkanes, as well as other hydrophobic groups, such as the fused-ring structure in cholic acid as found in bile salt surfactants, or phenyl groups that form a portion of the apolar group in Triton-type surfactants, and oligomer and polymer chains that nun the gamut from polyethylene (which represents a long alkane chain) to hydrophobic polymers, such as hydrophobic polypeptide chains in novel peptide-based surfactants that have been investigated.

Amphiphile: an amphiphile can be defined as a compound that contains both a hydrophilic and a lipophilic group. It is important to note that not every amphiphile is a surfactant. For example, butanol is an amphiphile, since the butyl group is lipophilic and the hydroxyl group hydrophilic, but it is not a surfactant since it does not satisfy the definition. given below. There exist a great many amphiphilic molecules possessing functional groups which are highly polar and hydrated to a measurable degree, yet. which fail to display surfactant behavior.

Surfactant: A surfactant is an amphiphile that possesses two additional properties. First, it significantly modifies the interfacial physics of the aqueous phase (at not only the air-water but also the oil-water and solid-water interfaces) at unusually low concentrations compared to nonsurfactants. Second, surfactant molecules associate reversibly with each other (and with numerous other molecules) to a highly exaggerated degree to form thermodynamically stable, macroscopically one-phase, solutions of aggregates or micelles. Micelles are typically composed of many surfactant molecules (10′s to 1000′s) and possess colloidal dimensions. Thus, in the present context, any amphiphile which at very low concentrations lowers interfacial tensions between water and hydrophobe, whether the hydrophobe be air or oil, and which exhibits reversible self-association into nanostructured micellar, inverted micellar, or bicontinuous morphologies in water or oil or both, is a surfactant. The term “lipids”, for all practical purposes. refers to a subclass of surfactants which are of biological origin.

Polar-apolar interface: In a surfactant molecule, one can find a dividing point (or in some cases, 2 points, if there arc polar groups at each end, or even more than two, as in lipid A, which has seven acyl chains and thus seven dividing points per molecule) in the molecule that divide the polar part of the molecule from the apolar pan. In any nanostructured liquid phase or nanostructured liquid crystalline phase, the surfactant forms monolayer or bilayer Films; in such a film, the locus of the dividing points of the molecules describes a surface that divides polar domains from apolar domains. this is called the “polar-apolar interface,” or “polar-apolar dividing surface.” For example, in the case of a spherical micelle, this surface would be approximated by a sphere lying inside the outer surface of the micelle, with the polar groups of the surfactant molecules outside the surface and apolar chains inside it. Care should be taken not to confuse this microscopic interface with macroscopic interfaces, separating two bulk phases, that are seen by the naked eye.

Bicontinuous: In a bicontinuous structure, the geometry is described by two distinct, multiply-connected, intertwined subspaces each of which is continuous in all three dimensions; thus, it is possible to traverse the entire span of this space in any direction even if the path is restricted to one or other of the two subspaces. In a bicontinuous structure, each of the subspaces is rich in one type of material or moiety, and the two subspaces are occupied by two such materials or moieties each of which extends throughout the space in all three dimensions.

Nanoporous: A material or phase, including a liquid or liquid crystalline phase. is nanoporous if it contains a system of nanometer-scale pores filled with water or other polar solvent (or mixture thereof), defined by porewalls that. can be solid or fluid, but that provide a barrier to diffusion of certain molecules, in particular enzymes and other macromolecules. In particular, a lipid bilayer can provide a porewall, since the diffusion of a macromolecule across a lipid bilayer is quite generally very slow compared to the diffusion of the same molecule in water. The diameter of a representative pore should be in the range of about I to 100 nm in order for the material or phase to be considered nanoporous.

Chemical criteria: In the case of surfactants, a number of criteria have been tabulated and discussed in detail by Robert Laughlin (See, Laughlin, Advances in Liquid Crystals, Vol. 3, p.41. 1978) for determining whether a given polar group is functional as a surfactant head group, where the definition of surfactant includes the formation, in water, of nanostructured phase,% even at rather low concentrations.

The following listing given by Laughlin gives some polar groups which are not operative as surfactant head groups, and thus. for example, an alkane chain linked to one of these polar groups would =be expected to form nanostructured liquid or liquid crystalline phase, are: aldehyde, ketone, carboxylic ester, carboxylic acid, isocyanate, amide, acyl cyanoguanidine, acyl guanylurea, acyl biuret, N,N-dimethylamide, nitrosoalkane, nitroalkane, nitrate ester, nitrite ester, nitrone, nitrosamine, pyridine N-oxide, nitrile, isonitrile, amine borane, amine haloborane, sulfone, phosphine sulfide, arsine sulfide, sulfonamide, sulfonamide methylimine. alcohol (monofunctional), ester (n)monofunctional), secondary amine, tertiary amine, mercaptan, thioether, primary phosphine, secondary phosphine, and tertiary phosphine.

Some polar groups which are operative as surfactant head groups, and thus, for example, an alkane chain linked to one of these polar groups would be expected to form nanostructured liquid and liquid crystalline phases. are:
a. Anionics: carboxylate (soap), sulfate, sulfamate, sulfonate. thiosulfate, sulfinate, phosphate, phosphonate, phosphinate, nitroamide, tris(alkylsulfonyl)methide, xanthate,
b. Cationics: ammonium, pyridinium, phosphonium, sulfonium, sulfoxonium;
c. Zwitterionics: ammonio acetate, phosphoniopropane sulfonate, pyridinioethyl sulfate, glycerophosphocholine;
d. Semipolars: amine oxide, phosphoryl. phosphine oxide, arsine oxide. sulfoxide, sulfoximine, sulfone diimine, ammonio amidate.

In addition to the polar head group, a surfactant requires an apolar group, and again there are guidelines for an effective apolar group. For alkane chains, which are of course the most common, if n is the number of carbons, then n must be at least 6 for surfactant association behavior to occur. although at least 8 or 10 is the usual case. Interestingly octylamine, with n =8 and the amine head group which is just polar enough to be effective as a head group, exhibits a lamellar phase with water at ambient temperature, as well as a nanostructured L2 phase. Branched hydrocarbons yield basically the same requirement on the low n end; for example, sodium 2-ethylhexylsulfate exhibits a full range of liquid crystalline phases. However, the two cases of linear and branched hydrocarbons are vastly different on the high n side. With linear, saturated alkane chains, the tendency to crystallize is such that for n greater than about 18, the Kraft temperature becomes high and the temperature range of nanostructured liquid and liquid crystalline phases increases to high temperature-, near or exceeding 100IC; in the context of the present invention, for most applications this renders these surfactants considerably less useful than those with n between 8 and 18. With the introduction of unsaturation or branching in the chains, the range of n can increase dramatic-ally. The case of unsaturation can be illustrated with the case of lipids derived from fish oils. where chains with 22 carbons can have extremely low melting points. due to the presence of as many as 6 double bonds, as in docosahexadienoic acid and its derivatives, which include monoglycerides, soaps, etc. Furthermore, polybutadiene of very high MW is an elastomeric polymer at ambient temperature, and block copolymers with polybutadiene blocks are well known to yield nanostructured liquid crystals. Similarly, with the introduction of branching, one can produce hydrocarbon polymers such as polypropyleneoxide (PPO), which serves as the hydrophobic block in a number of amphiphilic block copolymer surfactants of great importance. such as the Pluronic series of surfactants. bus discussed elsewhere, other hydrophobic groups, such as the fused-ring structure in the cholate soaps (bile salts), also serve as effective apolar groups. although such cases must generally be treated on a case by case basis, in terms of determining whether a particular hydrophobic group will yield surfactant behavior.

The invention is focused on a chemical segregating or separating device which can be used for chemical separations. assays, (drug delivery, and in other applications which includes a porous nanostructured liquid or liquid crystalline particle or material that is present in a reversed bicontinuous cubic phase, reversed hexagonal phase, L3 phase, normal bicontinuous cubic phase, or normal hexagonal phase phases, as described above. and a target which binds at least one chemical with specificity located in the porous. nanostructured liquid or liquid crystalline particle or material, where the target is accessible by the chemical of interest by diffusing in the porous nanostructured liquid or liquid crystalline particle or material. As discussed above, the chemical segregating or separating devices may be dehydrated variants of porous nanostructured liquid or liquid crystallin materials which include the target, whereupon reconstitution with water. blood, urine, mucuous or other fluid yields the porous nanostructured liquid or liquid crystalline particle or material.

In the practice of this invention, the target is preferably an antibody, receptor, chimera, lectin, nucleic acid sequence, or a fragment, simulant or derivative thereof. As discussed above. a fragment is a portion of the antibody, receptor, chimera, Pectin or nucleic acid sequence which retains the specific binding capacity of the base compound (e.g., a B fragment of an antibody). A simulant or derivative should he understood to be any compound that is deliberately produced or selected for its ability to hind, with a selectivity that is consistent with the desired functionality of the system, the analyte of interest. Typically. the search for such a compound begins with the naturally occurring target or targets. usually an antibody, lectin. receptor. or nucleic acid sequence, and extracts or mimics the critical binding regions or epitopes of the target.

The target will bind at least one chemical with specificity. Fragments, simulants and derivatives are variations on the antibody, receptor, chimera, lectin. nucleic acid sequence, that retain the ability to bind the same chemical with specifity as the base antibody, receptor, chimera, lectin. nucleic acid sequence. For example, a B fragment of an antibody will bind the same chemical (e.g., antigen, analyte, chemical of interest) as the complete antibody. Simulants of antibodies could have a similar, but not identical amino acid structure, or would otherwise be configured so as to hind the same chemical as the antibody they are simulating, Derivatives can be salts, ethers, esters, and the like, of the antibody, where the additional moieties (lo not destroy the binding capacity of the antibody derivative. Similar requirements exist for fragments, simulants and derivatives of receptors, chimera, lectin, and nucleic acids. The target moiety is bound in the porous nanostructured liquid or liquid crystalline particle or material (e.g., in the cubic phase) often by hydrophobic interaction. but less commonly by pore size or polymerization.

This invention has particular application to assays (e.g., competitive binding assays, sandwich assays, etc.) In an assay application of the invention, the chemical of interest is often referred to as an “analyte”. The analyte could be, for example, a hormone, neurotransmitter, peptide, protein, antibody, soluble receptor, virus, nucleic acid, endotoxin, microbial product, a specific sugar. drug molecule, or any of the compounds that arc screened for in a relevant diagnostic Assay or pharmaceutical screen, or a degradation product of any of the above.

In the case of a competitive assay. the molecule or chemical group that is originally bound to the Target, and is displaced by the Analyte is often referred to as a “Ligand”. The Ligand will generally be conjugated (attached) to either an Enzyme or Marker, or to an Enzyme or Marker through a series or other intermediates, such as biotin and avidin. In the case of a sandwich assay. this Ligand may not be needed. The ligand will be bound, either covalently or via other strong interactions such as those between avidin and biotin, to either the Enzyme or the Marker (described below). For the maximum flexibility in the system and ease of substitution or change in Analyte, a preferred setup is to have the Ligand covalently bonded to biotin, and the Enzyme (or less commonly, Marker) conjugated to avidin or streptavidin: in this way the Ligand is bound to the Enzyme (or Marker) through the intermediary avidin-biotin binding, and the ligand-Enzyme “train” can be changed without having to covalently bond the new Ligand to the Enzyme (in view of the fact that a great many l igands are commercially available as conjugates with biotin).

A “Marker” is a compound that, in response to action by an Enzymes or by other means, undergoes a measurable change, such as a change in color, or absorbance, or fluorescence, fluorescence decay, or luminescence, or specific conductance. etc. The preferred mode involves a change that is readily detected with a standard UV-Vis spectrometer, namely absorbance in the UV or visible range, preferably in the 200-1200 nm range, and more preferable in the range 400-900 nm. In a preferred embodiment of the invention. the assays arc colorimetric such that when a compound of interest is bound to the target, a marker provides a colorimetric change which can readily observed by a technician, clinician, or other individual.

An Enzyme in the context of assays according to the present invention contemplates a compound that reacts with the Marker to cause a measurable change. Preferably, this would be a compound that would traditionally be deemed an “enzyme” (e.g., a protein compound that catalyzes a change in a specific compound at a specific site), but, in the context of this invention should be more broadly understood to include any reactive compound, such us a catalyst or redox agent or otherwise reactive compound. that causes a measurable change with the Marker. hi competitive assays, either the enzyme will diffuse out of the porous nanostructured liquid or liquid crystalline particle or material to interact with the marker, or the marker will diffuse out of the porous nanostructured liquid or liquid crystalline particle or material to interact with the enzyme. However, in some embodiments, the enzyme would be designed to diffuse from the medium under test, into the porous nanostructured liquid or liquid crystalline particle or material.

In “sandwich assays”, a “second target” will be employed that is a compound, usually an antibody (preferable polyclonal) or nucleic acid sequence, that binds to the Analyte even when the Analyte is bound to the Target. The Second Target is conjugated to either the Marker, or more preferably, to the Enzyme.

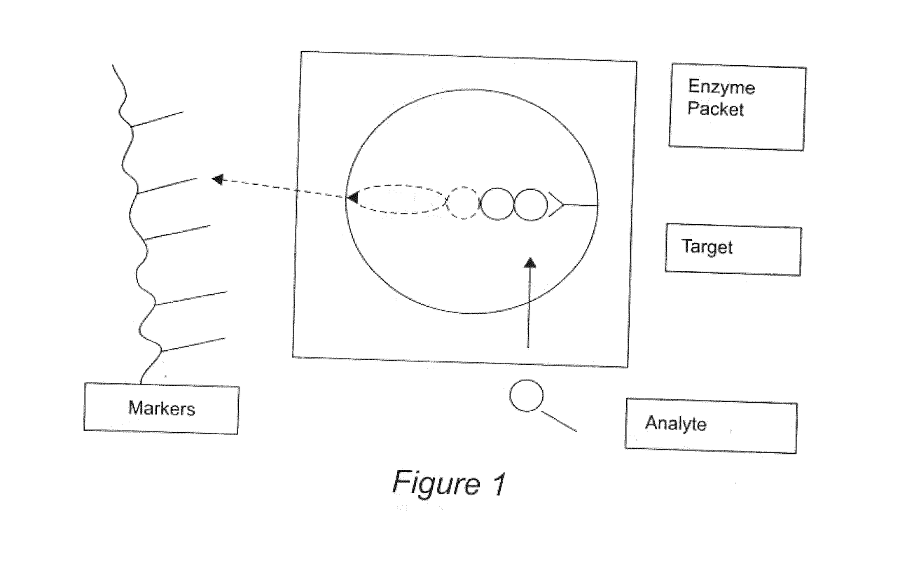

FIG. 1 illustrates one embodiment of the invention wherein an analyte in a medium diffuses into a porous, nanostructured lyotropic particle or material having a target retained therein, and where competitive displacement causes release of an enzyme packet from the lyotropic particle or material which then diffuses to the medium and interacts with markers bound to a substrate. polymer or the like which is too large to diffuse within the lyotropic particle or material. In a thin layer chromotography type application the substrate could be a paper material. The substrate might also be a biological “chip”, e.g., a silicon substrate on which chemical assays arc performed.

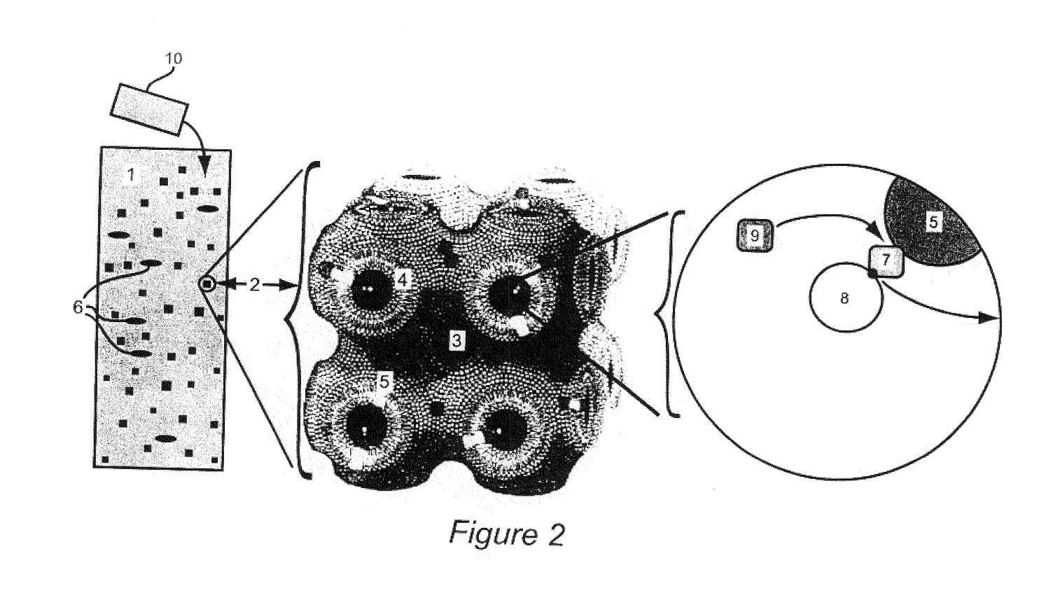

FIG. 2 illustrates in more detail aspects of the instant invention. In particular, a dispersion of particles I according to the invention is present in the medium 10 to be analyzed. This can be accomplished simply by combining the particles with the medium 10 and acting on the medium (e.g., agitation, stirring, etc.) to disperse the particles I therein, should such action be required. For purposes of this example, a particle 2 of the dispersion I takes the form of a reversed cubic bicontinuous phase. This particle 2 includes a aqueous pores 3 and a lipid bilayers 4 within the nanostructured of the particle 2. A target 5 is associated with the lipid bilayer 4, which is shown for exemplary purposes as a membrane spanning protein. Also included within the dispersion 1 is a marker 6 which is excluded from the interior of the particle 2. This can he accomplished by having the marker be of a molecular weight or chemical constitution that will not defuse into the nanostructure particle. Alternatively. the marker 6 could be adhered to a substrate, such as a bead within a cuvette which contains the dispersion 1. Preferably, a ligand 7 is bound to target 5 together with an enzyme or activator 8. The analyte 9 diffuses into the particle aid displaces the ligand 7 by binding with the target 5. This causes the enzyme or activator 8, with or without the ligand 7, to diffuse out of the particle I and interact with the marker 6. Thus, for the embodiment shown in FIG. 2, the material 10 to be analyzed is added to the dispersion 1, and, if analyte 9 is present. it will displace the enzyme packet comprising the ligand 7 and enzyme 8, so that the enzyme packet can diffuse out of the particle 2 through the pores 3, and make contact with the marker 6, causing a measurable change, for example in color or absorbance.

The dispersion of particles employed within the context of this invention, whether for assays or chemical sequestration, are preferably of low turbidity prior to binding of the chemical of interest to the target. By low turbidity, it should be understood that the dispersion is almost clear and that the dispersions absorbance at wavelengths ranging from about 300 nm to about 750 nm is less than about I absorbance unit, and preferably about 0.5 units, and most preferably less than about 0.3 absorbance units. Generally this requires that the particle size be less than about 300 nm. or preferably less than about 200 run, and more preferably in the range of 30 to 150 nm. Particle sizes in this preferred range permit low turbidity dispersions to be made it fairly high particle concentrations, e.g., greater than about 0.1% by volume.

The invention can be used in a number of different types of diagnostic assays. Table 1 below provides a summary of these assays. In the assays. the Target and Ligand are always inside the liquid crystal. The target can be hound inside the cubic phase either by hydrophobic interaction, poresize entrapment, covalent bonding to a liquid crystal component, or attachment to a solid dispersed within the liquid crystal. In Types H and V, the Marker can be bound inside the liquid crystal either by hydrophobic interaction, or covalent attachment, or attachment to a dispersed solid phase, or by poresize entrapment-in particular, by crosslinking of the Marker in the pores of a liquid crystal. as in U.S. Pat. No. 5,238,613. For sandwich assays in the context of Table 1. the “2 Target-Enzyme” means the Second Target conjugated to the Enzyme, and “C or S” means Competitive or Sandwich assay.

TABLE 1

| | Inside | Outside | Mode of operation |
|---|---|---|---|
| TYPE | the L.C. | the L.C. | C or S |
| I | Enzyme | Marker | Analyte displaces Ligand, C |
| | | | Ligand-Enzyme diffuses out |
| | | | of L.C. and reacts with Marker |
| II | Marker | Enzyme | Analyte displaces Ligand, C |
| | | | Ligand-Marker diffuses out |
| | | | of L.C., and reacts with Enzyme |
| III | Neither | 2^{nd} Target- | Analyte binds to Target, S |
| | | Enzyme, | (Target only)then Marker added |
| | | | 2^{nd} Target-Enzyme enters |
| | | | L.C. and binds, Marker added |
| | | | and reacts only with unbound |
| | | | Enzyme in the exterior phase |
| IV | Neither | 2^{nd} Target- | Analyte binds to Target, S |
| | | Marker, | (Target only)then Enzyme added |
| | | | 2^{nd} Target-Marker enters |
| | | | L.C. and binds, Enzyme added |
| | | | and reacts only with unbound |
| | | | Marker in the exterior phase |
| V | Marker | 2^{nd} Target- | Analyte binds to Target, then |
| | | Enzyme | add S (added after Analyte) |
| | | | 2^{nd} Target-Enzyme, which |
| | | | enters L.C. to bind Analyte, so |
| | | | reaction between Marker and |
| | | | Enzyme accelerates |

The preferred mode of operation when the Analyte is a small molecule is Type I; this gives amplification of the signal, since a single displacement can release an enzyme that can cause many reactions on the Marker. F-or the case where the Analyte is a protein. the preferred mode of operation is Type V since the displacement of a protein is often not efficient whereas sandwich assays are well established, and because in Type V, there is a low reaction rate unless the Analyte is present.

For the case where the Analyte is a nucleic acid (RNA or DNA), the preferred approach would be to choose a Target that would be a complementary strand to a single-stranded form of the Analyte; embodiments and variations of this single-strand approach to selective binding are well known in the art. This could be bound inside the liquid crystal by a hydrophobic anchor, by poresize entrapment, by an antibody, or more preferably by covalent attachment to a bilayer component or solid dispersed in the liquid crystal. Then in the case where the MW of the single strand is not prohibitively high (possibly involving the application of nucleases), Type I approach could be used: a weakly hound nucleic acid conjugated to an Enzyme would be bound to the Target, and this would be displaced by the Analyte since the latter would bind more strongly to the Target. If, on the other hand, the MW of the Ligand that would be required (to achieve the proper specificity) were too high for displacement to be practical, then Type V methodology could be applied: in this case. the Target and the “Second Target” could in fact he two nucleic acids with sequences that are complementary to two (preferably non-overlapping) sequences within the Analyte.

The assays described above may be used for testing for chemicals in almost any tyke of media including blood, urine, saliva, aqueous media, oil based media, etc. They may also be able to be used on solids, e.g., the skin, eye, genitals tongue. etc. where chemicals are transported and diffuse into the porous nanostructured liquid or liquid crystalline particles or materials. These applications might best employ a paper, metal, plastic or other substrate.

As discussed above,

The nanostructured liquid phase material may be formed from:

a. a polar solvent and a surfactant or

b. a polar solvent, a surfactant and an amphiphile or hydrophobe or

c. a block copolymer or

d. a block copolymer and a solvent.

The nanostructured liquid crystalline phase material may be formed from:

a. a polar solvent and a surfactant.

b. a polar solvent, a surfactant and an amphiphile or hydrophobe. or

c. a block copolymer or

d. a block copolymer and a solvent.

Polar and apolar groups are preferably selected in order lo make an operative surfactant Thus, suitable surfactants include those compounds which contain two chemical moieties. One being an operative polar group chosen from those described in that discussion of polar groups, and the other being an operative apolar group chosen from those described in that discussion of apolar groups.

Suitable surfactants or block copolymer components or mixtures thereof may include:

a. cationic surfactant

b. anionic surfactant

c. semipolar surfactant

d. zwvitterionic surfactant
i. in particular, a phospholipid
ii. a lipid mixture containing phospholipids, designed to match the physico-chemical characteristics of a biomembrane

e. monoglyceride

f. PEGylated surfactant

g. one of the above but with aromatic ring

h. block copolymer
i. with both blocks hydrophobic, but mutually immiscible
ii. with both blocks by hydrophilic, but mutually immiscible.
iii. with one block hydrophilic and the other hydrophobic. i.e., amphiphilic)
iv. a mixture of two or more of the above.

Suitable lipids include phospholipid.s (such as phosphatidylcholine. phosphatidylserine, phosphatidylethanolamine, or sphingoimyelin), or glycolipids (such as MGDG, diacylglucopyranosyl glycerols, and Lipid A.) Other suitable lipids are phospholipids (including phoqphatidylcholines, phosphatidylinositols, phosphatidylglycerols, phosphatidic acids, phosphatidylserines. phosphatidylethanolamines, etc.), sphingolipids (including sphingomyelins), glycolipids (such as galactolipids such as MGDG and DGDG, diacylglucopyranosyl glycerols, and Lipid A), salts of cholic acids and related acids such as deoxycholic acid, glycocholic acid, taurocholic acid, etc., gentiobiosyls. isoprenoids, ceramides, plasmologens, cerebrosides (including sulphatides), gangliosides. cyclopentatriol lipid., dimethylaminopropane lipids, and lysolecithins and other lysolipids which are derived from the above by removal of one acyl chain.

Other suitable types of surfactants include anionic. cationic, zwittenionic, semipolar, PEGylated, and amine oxide. Preferred surfactants are:
anionic—sodium oleate, sodium dodecyl sulfate, sodium diethylhexyl sulfosuccinate. sodium dimethylhexyl sulfosuccinate. sodium di-2-ethylacetate, sodium 2-ethylhexyl sulfate, sodium undecane-3-sulfate, sodium ethylphenylundecanoate, carboxylate soaps of the form ICₙ, where the chain length n is between 8 and 20 and I is a monovalent counterion such as lithium, sodium, potassium. rubidium. etc.
cationic—dimethylammonium and trimethylammonium surfactant. or chain length from 8 to 20 and *with chloride. bromide or sulfate counterion. myristyl-gammapicolinium chloride and relatives with alkyl chain lengths from 8 to 18, benzalkonium benzoate, double-tailed quaternary ammonium surfactants with chain lengths between 8 and 18 carbons and bromide. chloride or sulfate counterions. nonionic PEGylated surfactants of the form Cₙ,Eₘ where the alkane chain length n is from 6 to 20 carbons and the average number of ethylene oxide groups m is fiom 2 to 80, ethoxylated cholesterol;

zwitterionics and semipolar—N,N,N-trimethylaminodecanoimide, amine oxide surfactants with alkyl chain length from 8 to 18 carbons;

dodecyldimethylammoniopropane-1-sulfate, dodecyldimethylammoniobutyrate, dodecyltrimethylene di(ammonium chloride); decylmethylsulfonediimine;

dimethyleicosylammoniohexanoate and relatives of these zwitterionics and semipolars with alkyl chain lengths from 8 to 20.

Preferred surfactants which are FDA-approved as injectables include phospholipids (particularly phosphatidylcholine), benzalkonium chloride. sodium deoxycholate, myristyl-gamma-picolinium chloride, Poloxamer 188, polyoxyl castor oil (including Cremophor and certain other ethoxylated derivatives of castor oil), sorbitan monopalmitate and sodium 2-ethylhexanoic acid. It is especially useful in certain embodiments to form a bicontinuous, cubic phase using phosphatidylcholine and water (or other polar solvent, especially glycerol), wherein a third component is required. which can he, for example, one of the following compounds: an essential oil (preferred oils being oils of ginger, santalwood, cedarwood, patchouli. peppermint, carrot seed, cloves, ylang-ylang, fir needle. mugwort, oregano, chamomile, eucalyptus, thuja, hyssop, spearmint and myrth, with ginger, cloves, and ylang-ylang being especially preferred, as well as components of these oils), Vitamin E, oleoresins (such as those of capsaicin), long-chain unsaturated alcohol,; and fatty acids (arid long-chain unsaturated compounds with other polar groups, such as amines, etc.), tryptophan, proteins such as casein or albumin, sorbitan triacyl esters, and docusate salts. Other preferred lipids include glycerol monooleate (or other long-chain unsaturated monoglycerides), Arlatone G, Tween 85, Caprol, didodecyldimethylammonium bromide, and Pluronic 123 and other low-HLB Pluronics and Tetonics.

Lipids and surfactants that are of low toxicity and also low water-solubility are especially preferred in certain applications of this invention, such as those in which the particles are implanted ill, or administered to, a mammal, and include: acetylated monoglycerides, aluminum monostearate, ascorbyl palmitate free acid and divalent salts, calcium stearoyl lactylate, ceteth-2, choleth, deoxycholic acid and divalent salts, dimethyldioctadecylammonium bentonite, docusate calcium, glyceryl stearate, stearamidoethyl diethylamine, ammoniated glycyrrhizin, lanolin nonionic derivatives, lauric myristic diethanolamide, magnesium stearate, methyl gluceth- 120 dioleate, monoglyceride citrate. octoxynol-1, oleth-2. oleth-5, peg vegetable oil, peglicol-5-oleate, pegoxol 7 stearate. poloxamer 33 1, polyglyceryl-10 tetralinoleate, polyoxyethylene fatty acid esters, polyoxyl castor oil, polyoxyl distearate, polyoxyl glyceryl stearate, polyoxyl lanolin, polyoxyl-8 stearate, polyxyl 150 distearate. polyoxyl 2 stearate, polyoxyl 35 castor oil. polyoxyl 8 stearate, polyoxyl 60 castor oil, polyoxyl 75 lanolin, polysorbate 85, sodium stearoyl lactylate, sorbitan sesquioleate, sorbitan trioleate, stear-o-wet c, stear-o-wet m, stearalkonium chloride, stearamidoethyl diethylamine (vaginal), steareth-2. steareth-10, Stearic acid, stearyl citrate, sodium stearyl fumarate or divalent salt, trideceth 10, trilaneth4 phosphate, detaine ph, jbr204 rhamnolipid (from Jeneil Biosurfactant). glycocholic acid and its salts. taurochenodeoxycholic acid (particularly combined with vitamin E), tocopheryl dimethylaminoacetate hydrochloride, tocopheryl phosphonate, tocopheryl peg 1000 succiniate, cytofectin gs, 1,2-dioleoyl-sn-glycero-3-trimethylammonium-propane. cholesterol linked to lysinamide or ornithinamide, dimethyldioctadecyl ammonium bromide, I,2-dioleoyl-sn-3-ethylphosphocholine and other double-chained lipids with a cationic charge carried by a phosphorus or arsenic atom, trimethyl aminoethane carbamoyl cholesterol iodide, lipoic acid, O,O′-ditetradecanoyl-n-(alpha-trimethyl ammonioacetyl) diethanolamine chloride (DC-6-14), N-[(1-(2,3-dioleyloxy)propyl)]-N-N-N-trimethylammonium chloride, N-methyl-4-(dioleyl)methylpyridiniumchloride (saint-2), lipidic glycosides with amino alkyl pendent qroups, 1,2-dimyristyloxypropyl -3-dimethylhydroxyethyl ammonium bromide, bis[2-(11-phenoxyundecanoate)ethyl]- dimethylamnionium bromide, N-hexadecyl-N-10-[O-(4-acetoxy)-phenylundecanoate]ethyl-dimethylammonium bromide, bis[2-(11-butyloxyundecanoate)ethy]dimethylammonium bromide, 3-beta-[N-(N′,N′-dimethylaminoethane)-carbamoyl] cholesterol, vaxfectin, cardiolipin, dodecyl-n,n-dimethylglycine, and lung surfactant (Exosurf, Survanta).

Suitable block copolymers are those composed of two or more mutually immiscible blocks from the following classes of polymers: polyienes, polyallenes, polyacrylics and polymethacrylics (including, polyacrylic acids, polymethacrylic acids, polyacrylates, polymethacrylates, polydisubstituted esters, polyacrylamides, polymethacrylamides, etc.), polyvinyl ethers, polyvinyl alcohols, polyacetals, polyvinyl kelones, polyvinylhalides, polyvinyl nitriles, polyvinyl esters, polystyrenes, polyphenylenes, polyoxides, polycarbonates, polyesters, polyanhydrides, polyurethanes, polysulfonates, polysiloxane, polysulfides, polysulfones, polyamides, polyhydrazides, polyureas, polycarbodiimides, polyphosphazenes, polysilanes, polysilazanes, polybenzoxazoles, polyoxadiazoles, polyoxadiazoiidinies, polythiazoles, polybenzothiazoles, polypyromellitimides, polyquinoxalines, polybenzimidazoles, polypiperazines, cellulose derivatives, alginic acid and its salts, chitin, chitosan, glycogen, heparin, pectin, polyphosphorus nitrile chloride, polytri-n-butyl tin fluoride, polyphosphoryldimethylamide, poly.-2,5-selenienylene, poly4-n-butylpyridinium bromide, poly-2-N-methylpyridinium iodide, polyallylammonium chloride, and polysodium-sulfonate-trimethylene oxyethylene. Preferred polymer blocks are polyethylene oxide, polypropylene oxide, polybutadiene, polyisoprene, polychlorobutadiene, polyacetylene, polyacrylic acid and its salts, polymethacrylic acid and its salts, polyitaconic acid and its salts, polymethylacrylate, polyethylacrylate, polybutylacrylate, polmethylmethacrylate, polypropylmethacrylate, poly-N-vinyl carbazole, polyacrylamide, polyisopropylacrylamide, polymethacrylamide, polyacrylonitrile, polyvinyl acetate, polyvinyl caprylate, polystyrene, poly-alpha-methylstyrene, polystyrene sulfonic acid and its salts, polybromostyrene, polybutyleneoxide, polyacrolein, polydimethylsiloxane, polyvinyl pyridine, polyvinyl pyrrolidone, polyoxy-tetramethylene, polydimethylfulvene, polymethylphenylsiloxane, polycyclopentadienylene vinylene, polyalkylthiophene, polyalkyl-p-phenylene, polyethylene-alt-propylene, polynorbomene, poly-5-((trimethylsiloxy)methyl)norbomene, polythiophenylene, heparin, pectin, chitin, chitosan, and alginic acid and its salts. Especially preferred block copolymers are polystyrene-b-butadiene, polystyrene-b-isoprene, polystyrene-b-stenesulfonic acid, polyethylenoxide-b-propyleneoxide, polystyrene-b-dimethylsiloxane, polyethyleneoxide-b-styrene, polynorbornene-b-5-((trimethylsiloxy)methyl)norbornrne, polyaecetylene-b-5-((trimethylsiloxy)methyl)norbornene, polyacetylene-b-norbornene, polyethyleneoxide-b-norbornene, polybutyleneoxide-b-ethyleneoxide, polyethyleneoxide-b-siloxane, and the triblock copolymer polyisoprene-b-styrene-b-2-vinylpyridine.

A third component, such hydrophobe or non-surfactant amphiphile, may also be included in the porous nanostructured liquid or liquid crystalline phase particles or materials, such as a:
a. alkane or alkene, other long-chain aliphatic compound
b. aromatic compound, such as toluene
c. long-chain alcohol
d. glyceride (diglyceride or triglyceride)
e. acylated sorbitan, such as a sorbitan triester (e.g., sorbi trioleate), or sesquioleate, or mixture of sorbitans with different numbers of acyl chains between 2 and 6
f. other hydrophobe or non-surfactant amphiphile or mixture with one or more of the above,
g. none.

Suitable third components (hydrophobes or non-surfactant amphiphiles), include: n-alkane, where n is from 6 to 20. including branched, unsaturated, and substituted variants (alkenes, chloroalkanes, etc.), cholesterol and related compounds, terpenes, diterpenes, triterpenes, fatty alcohols, fatty acids, aromatics, cyclohexanes, bicyclics such as naphthalenes and naphthol, quinolines and benzoquinolines, etc., tricyclics such as carbazole, phenothiazine, etc., pigments, chlorophyll, sterols, triglycerides, natural oil extracts (such as clove oil, anise oil, cinnamon oil, coriander oil, eucalyptus oil, peppermint oil), wax, bilirubin, bromine, iodine, hydrophobic and amphiphilic proteins and polypeptides (including gramicidin, casein, receptor proteins, lipid-anchored proteins, etc.), local anesthetics (such as butacaine, ecgonine, procaine, etc.), and low-molecular weight hydrophobic polymers (see listing of polymers above). Especially preferred third components are: anise oil, clove oil, coriander oil, cinnamon oil, eucalyptus oil, peppermint oil, beeswax, benzoin, benzyl alcohol, benzyl benzoate, naphthol, capsaicin, cetearyl alcohol, cetyl alcohol, cinnamaldelhyde, cocoa butter, coconut oil, cottonseed oil (hydrogenated), cyclohexane, cyclomethicone, dibutyl phthalate, dibutyl sebacate, dioctyl phthalate, DIPAC, ethyl phthalate, ethyl vanillin, eugenol, fumaric acid, glyceryl distearate, menthol, methyl acrylate, methyl salicylate, myristyl alcohol, oleic acid, oleyl alcohol, benzyl chloride, paraffin, peanut oil, piperonal, rapeseed oil, rosin, sesame oil, sorbitan fatty acid esters, squalane, squalenie, stearic acid, triacetin, trimyristin, vanillin, and vitaniin E.

The polar solvent (or in the case of a block copolymer, the preferential solvent) may be:
a. water
b. glycerol
c. formamide, N-methyl formamide, or dimethylformamide
d. ethylene glycol or other polyhydric alcohol
e. ethylammonium nitrate
f. other non-aqueous polar solvents such as N-methyl sydnone, N-methyl acetaminde, pyridinium chloride, etc.;
g. a mixture of two or more of the above.

Desirable polar solvents are water, glycerol, ethylene glycol, formamide, N-methyl formamide, dimethylformamide, ethylammonium nitrate, and polyethylene glycol.

As noted above, antibodies are preferred bound, immobilized or retained entities within the porous nanostructured liquid or liquid crystalline particles or materials of this invention for assays and other applications. There are a wide variety of commercially available antibodies which may be useful in the practice of this invention including: 8-hydroxy-guanosine, AAV (adeno virus), ACHE (acetylcholinesterase), ACHER (acetylcholine and NMDA receptor), acid phosphatase, ACTH, Actin (cardiac, smooth muscle, and skeletal), Actinin, Adeno-associated virus, adenosine deaminase, Adipophilin (adipocy differentiation related peptide), Adrenomedulin 1-6, Advanced glycation end-products (AGE), alanine transaminase, albumin, alcohol dehydrogenase, aldehyde dehydrogenase, aldolase, Alfentanil AB, Alkaline Phosphatase, alpha Actinin. Alpha-1-anti-chymotrypsin, alpha-1-antitrypsin, alpha-2-macroglobulin, alpha-catenin, beta-catenin and gamma cateinin, Alpha-Fetoprotein, Alpha-fetoprotein receptor, Alpha-Synuclein, Alzheimer Precursor Protein 643-695(Jonas), Alz-90, Precursor Protein A4, amino acid oxidase, Amphetamine, amphiphysin, amylase, amylin, Amylin Peptide, Amyloid A and P, Amyloid precursor protein, ANCA (Proteinase PR3), androgen receptor, Angiogenin, Angiopoietin-1and Angiopoietin-2 (ang-1/Ang-2). Angiotensin Converting Enzyme, Angiotensin II Receptor At1 and At2, Ankyrin, Apolipoprotein D, Apolipoprotein E, arginase 1, B Arrestin 1 and B Arrestin 2, ascorbate oxidase, asparaginase, aspartate transaminase, Atpase (p97), atrial Natriuretic Peptide, AU1 and AU5, Bacillus Antracis (Anthrax) and Bacill, antracis lethal factor, Bad, BAFF, Bag-1, BAX, bel-2, BCL-XI, B Nerve Growth Factor, BETA Catenin, Benzoylecognine (cocaine), beta-2 microglobulin, Beta Amyloid, Galactosidase, Beta Glucuronidase, Blood Group antigens (RhoD, A1,A2 A1, A2, A3, B, A, Rh(0)D, RhoC, B M, N), Blood Croup H antigen, bombesin and bombesin/gastrin releasing peptide, Bone Morphogenetic Protein (BMP), Bone marrow stromal cell antigen, BST-3. Borrelia burgdorferi garinii, borrelia burgdorferi sensustricto, Bovine Serum, Bradykinin Receptor B2, Brain derived neutrophic factor, Bromodeoxyuridine, CA 19-9, CA 125, CA 242, CA 15-3, CEA, Ca+ATPase, Calbindin D-28K (Calcium binding protein), Calgranulin A, Cadherin, CD144, Calcineurin, Calcitonin, Calcitonin gene related peptide, Calcium Channel, Caldesmon, Calmodulin, Calnexin, Calpactin light chain, Calpain, Calpastatin, Calreticulin, Calretinin, Calsequestrin, Cam Kinase II. Canine Distemper virus, carbonic anhydrase I and II, Carboxypeptidase A, B and E, Carboxypeptidase Y, Cardi, Troponin C and T. cardiotirophin-1, Caspase 3 (CPP32), Catalase, Catenins, Caveolin 1, 2 a and 3, CCR, CD44 (HCAM), CD56 (NCAM), CDK2, CDK4 (Cyclin Dependent Kinase C), Carcinoembryonic Antigen, Cellular antigens, CFTR (cystic fibrosis transmembrane conductance protein), chemokine receptors, chlamydia, CHO cell (Chinese Hamster Ovary Cell) Proteins, cholera toxin, choline oxidase, Chondroitin, Chloramphenic, Acetyltransferase(CAT), Chromogranin A, B and C (Secreogranin III), cholesterol oxidase, Chymotrypsin, Cingulin, Citrate Synthethetase, C-kit/stem cell factor receptor, CK-MB, Clathrin Antigen, Clostridium Botulinum D Toxoid, Clusterin, C-MYC, CNS Glycoprotein 130kD, Collagen Type IV and Type VII, Complement 5b neocpitope, Complement C3a, C3b, C5 and C9, complexin 2, Corticoliberin (CRF), C-peptide, CRF (Corticotropin Releasing Factor), Corticotropin releasing factor receptor, COX-1 and Cox-2, CPP32 (also known as Caspase 3, apopain or Yama), Creatine transporter, C-Reactive Protein (CRP), Cryptosporidium, CXCR-5, Cyclin A, Cyclin D1, D2 and D3, Cyclosporine A, Cylicin I, Cytochrome B5, Cytochrome C, Cytochrome oxidase, Cytochrome P450, Cytokeratin Types I and II, Cytomegalovirus, DAP Kinase, Dentritic cells, Desmin, Desmocollin 1, 2 and 3, Desmoglein 1, 2 and 3, Desmoplakin 1 and 2. Dextranase, DHT (Dihydrotestosterone, Dihydrofolate Reductase (DHFR), Dioxin, Diptheria toxin, Distemper, DJ-1. DNA single-stranded, DNA double stranded, DNA Topoisomerase II and Phosphotopoisomerase IIa+IIb alpha/beta, Dopanline, Dopaminie Beta-Hydroxylase, Dopamine Receptor, Dopamine Transporter, Drebrin, Dysferlin, Dystrobrevin, E.Coli expression plasmid, Elastase, Elastin, Endocrine Granu, Constituent (ECC), Endorphin, Endothelial cell, Endothelin, Endothelin Receptor, Enkephalin, enterotoxin Staphylococcus aureus, Eosinophil Peroxidase, Eosinophil derived neuroitox, (EDN), Eotaxin, Eotaxin-2, Epidermal Growth Factor, epidermal growth factor receptor, testostosterone, Epithelial Proliferating antigen, Epithelium Specific Antigen, c-MYC, HA.1, VSV-G Tag, Glu-Glu, EEEYMPME, Thioredoxine (trx), Epstein Barr virus and Epstein Barr Virus capsid antigen gp120, ERK (ERK1, ERK2, ERK3, pan ERK also called MAP kinase), Erythrocytes, Erythropoietin (EPO), Esterase, Estradiol, Estriol, Estrogen Receptor, Estrone, Ets-1 transcription, F1 antigen Yersina pestis, Factor 5, Factor VII, Factor VIII, Factor 9, Factor 10. Factor 11, Factor 12, Factor XIII, FAK (Focal Adhesion Kinase), FAS (CD95), FAS-L (CD178), Fascin. Fatty Acid Binding Protein, Ferritin, Fetal Hemoglobin, Fibrillin-1, Fibrinogen, Fibroblasts, Fibroblast Growth Factor, FGF-9, Fibronectin, Filamin, FKBP51, FKBP65, FK506, FLK1, flt-1 FLt-4 and FLT-3/FLK-2, FLT 3 Ligand, Fluorescein (FITC), FODRIN, Folate, Folate Binding Protein, fractalkine, frequenin, Frizzled, Fructose-6-p-kina, FSH, Fusin (CXCR4), GABA A and GABA B Receptor, Galectin, galanin, gastrin, GAP-43, G-CSF, C-CSF receptor, gelsolin, GIP (gastric inhibitory peptide), G0-protein (bovine), GDNF, CDNF-Receptor, Giardia intestinalis, Glial fibrillary acidic Protein, Glial filament protein, Glucagon/Glycentin, Glucose oxidase, Glucose 6 Phosphate Dehydrogenase, Gluco, Tranporter GLUT 1-4, GLUT 1-5. Glutamate Dehydrogenase, Glutamic Acid decarboxyla (GAD), Glutathione, Glyceraldehyde-3-phosphate dehydrogenase GAPDH, Glycerol-3-phosphate dehydrogenase, Glycerol kinase, glycine transporter (GLYT1, GLYT2). Glycogen Phosphoralase Isoenzyme BB (GPBB), Glycophorin A (CD235a). GM-CSF, C receptor alpha, Golgi Complex, Gonadotropin-Releasing Hormone Receptor (GnRWR), GP130, Granzymne, GRB2, GRB1, Green Fluorescent Protein (GFP), Growth Hormone, Grow, Hormo, Receptor, Growth Hormone Releasing factor, CRP78, Hantavirus, HCG, HDL (high density lipoprotein), Heat Shock Protein HSP-27, HeK 293 Host Cell Proteins, Helodermin, helospectin, Hemeoxygenase, Hemoglobin, Heparin, Hepatitis A, Hepatitis B Core Antigen, Hepatitis B virus surface antigen, Hepatitis C vimis, Hepatistis E virus, Hepatitis G Virus, Hepatocyte Growth Factor, Heregulin (Neu differentiation factor/Neuregulin), Herpes Simplex Virus, Hexokinase, Histamine, His Tag, 6-His vector tags, HIV-1 p24, p55/17, gp41, gp120, tat, nef, rev, HIV reverse transcriptase, HLA Class I, HLA Class II, HLA-DM, HLA DQw1, HLA DRw 52, HorseRadish Peroxidase, HPV 16 Late I Protein, human free kappa light chains, human lambda light chains. Human IgA, human I heavy chain, human IgA1, human IgD, human IgE. human IgG heavy chain, human IgG1, human IgG3, human IgG4, human IgM, human lgM heavy chain, hunman J chain, human kappa lig, chains, hulnan lambda light chains, Human Serum Amyloid P, Human Serum Amyloid P, Interleukin 1 beta converting enzyme, ICH-1 (caspase 2). Indian Hedgehog Protein (IHH), Influenza virus, Inhibin, Insulin, insulin like growth factor II, insulin growth factor binding protein 1, 2, 3, 4 or 5, insulin like growLh factor, insulin like growth factor I receptor, insulin receptor, insulin/proinsulin, Interferon alpha, interferon alpha receptor, Interferon Beta, Interferon Gamma, interferon gamma receptor alpha and beta, Interleukin 1 alpha, Interleukin Receptor alpha type II, lnterleukin 1-beta, Interleukin 10, interlukin 10 receptor, Interleukin 11, Interleukin 12, Interleukin 12 receptor. Interleukin 13. interleukin 15. Interleukin 16, Interleukin 17, Interleukin 18, Interleukin 2, Interleukin 2 receptor alpha, Interleukin receptor alpha chain (CD25), lnterleukin 2 receptor beta, Interleukin 2 receptor beta chain(CD122). Interleukin 2 receptor gamma, Interleukin 3, Interleukin 3/interleukin 5/GM-CSF Receptor common chain, Interleukin 4. Interleukin 5. Interleukin 6, Interleukin 6 receptor alpha chain, Interleukin 7, Interleukin 7 receptor alpha, Interleukin 8, Interleukin 8 receptor, Interleukin 9. invertase, Involucrin, lP-10, Keratins. KGF, Ki67, KOR-SA3544, Kt3 epitope tag, lactate dehydrogenase, Lacktferrin, lactoperoxidase, Lamins, Laminin, La (SS-B). LCMV (Lymphocytic Choriomeningitis Virus), Legionella pneumophilia serotype, Legionella pneumophila LPS, Leptin and Leptin Receptor, Lewis A Antigen, LH (leutenizing Hormone), LHRH (leutenizing Hormone Releasing), L. (leukemia Inhibitory Factor). 5-lipoxygenase, LPS Francesella tularensis, luciferase, Cancer Marker (MOC-1, MOC-21, MOC-32, Moc-52). Lymphocytes, lymphotactin, Lysozyme, M13, F1 Filamentous Phages, Macrophages/monocytes, Macrophage Scaveng, Receptor, Matrix metalloproteases, M-CSF. Major Basic Protein, malate dehyrogenase, Maltose Binding Protein, Mannose Receptor (macrophage), Mannose-6-phosphate receptor. MAP kinase antibodies (ERK, ERK, ERK2, ERK3), MASH1 (Mammnalian achacte schute homolog 1 and 2), MCL-1, Mcm3, M, (MCAF), MCP-2, MCP-3, Melanocortin Receptors (1 through 5), Met (c-met), Mineralcortocoid Receptor (MR/MCR), Melanoma Associated Antigen, MGMT (methylguanine-DNA-methyltransferase), MHC Antibodies (incl. HLA DATA PACK), Milk F. Globule Membrane, Milk Mucin Core Antigen, MIP-1 alpha, MIP-1 beta, Mitochondrial markers, Mitosin, MMP-1, MM, MMP3. MMP7. MMP8. MMP-9 and MMP13 (matrix metalloproteases), MMP-14(MT1-MM, MMP15 (MT2-MMP). MMP16(MT3-MMP) and MMP19, Morphine, motili. Mucin related antibodies (Muc-1, muc-2, muc-3, muc-5ac), Mucin-6 glycoprotein, Mucin-like Glycoprotein, Mycobacterium tuberculosis, Myelin, Myelin Basic Protein, Myeloperoxidase, MyoD, Myoglubin, Myosin, Na+Ca+Exchanger Protein, Na+/K+/ATPase, Na+/K+/ATPa. NICAM (CD56), pan N-Cam. (neural cell adhesion marker), Nerve Growth Factor, Neu-Oncogene (c-crb B2), Neurofibrillary Tangle, Neurofilament 70+200 kD, Neurofilament. 145 Kd, neurofilament 160 kd. Neurofilament 68 Kd, Neurofilament 200 kd, Neurofilament 200 kd, neurokin, A/substance K, neuromedin U-8 (NMU-8). Neuromodulin, neuronal pentraxin, Neuro-Specific Enolase, Neuropeptide Y (NPY), Neurophysin I (oxytocin precursor). Neurophysin, (vasopressin precursor), Neuropsin, Neurotensin, NFKB. Nicotinic Acetylcholine Receptor, (Beta2 and Alpha 4), NMDA receptors, N-MYC, Norepinephrine Transporter (NET), N, (Nitric Oxide Syntase) cNos. iNos, NT-3, NT, (neurotroph. 4), Nucleolar Helicase, Nucleolar Protein N038. Nuclear Protein xNopp180, Nucleoplasm, Protein AND-1, Nucleolus Organizing Region (NOR), Nucleolin, occludin, Oncostatin M, ORC, Ornithine Decarboxylase, Ovalbumin, Ovarian Carcinoma, Oxytocin, P15, P16, P2, P27, P53 Oncoprotein, p62 Protein, p97 Atpase, membrane associated and cytosolic 42 kDa 1inositol (1,3,4,5) tetrakisphosphate receptor. PP44 Podocyte Protein (Synaptopodin), PAH (Polyaromatic Hydrocarbons), PACAP (pituitary adenylate cyclase activating peptide), Pancreas Polpeptide (PP), Pancreastatin, Pancreatic Islet Cell, papain, Papillomavirus (HPV), Parainfluenza type 2 viruses, Parathion, Parkin, PARP (Poly-A, Riobose Polymerase) PARP-1 and PARP-2, Patched-l1, Patched-2, Paxillin, polychlorinated biphenyls, Pemphigus vulgaris (desmoglein 3), Penicillin, penicillinase, pep-carboxylase, pepsin. Peptide YY, Perforin and polyclonals, Perilipin, Peripherin, Perlecan, Petrole, Hydrocarbons (total), PPAR (peroxisome proliferation activated receptors), P-Glycoprotein (multi-drug resistance), PGP9.5, Phenanthrene, Phencyclidine (PCP), Phenylethanolamine, methyltransferase (PNMT), Phospholamban, Phospholipase A2. Phosphoserine, Phosphothreonine, Phosphotyrosine, Phosphothreonine-proline, phosphothreonine-lysi, phophotyrosi, Phosphotyrosine Kinase, Pichia pastoris, Placent, Alkaline Phosphatase, Plakoglobin, Plakophilin 1, Plakophilin 2, Plakophilin 3, Plasminogen, Platelet Derived Growth Factor AA and BB and AB, Plectin, PM, ATPase (plasma membrane Ca pump), Pncumocystis carinii, Pneumolysin, Polychlorobiphenyl (PCB), PP17/TIP47. PPAR (peroxisome proliferation activated receptors). Prednisone, Prednisolone, Pregnancy associated Plasnia Protein A (PAPP-A), Pregnenolone, Prepro NPY 68-97, Presenilin-1, Presenilin-2. Prion protein, Progesterone, Progestcro, Receptor, Prohibitin, Proinsulin, Prolactin, Proliferation Ce, Nuclear Antigen, Proline Transporter, Prostatic Acid Phosphatase (PAP), Prostatic Specif, Antigen (PSA), Proteasome 26S. Protein 4.1 M ascites, Protein G, Protein Kinase C, Pseudomonas mallei, PTH, Pulmonary Surfactant Associated Proteins, Puromycin, Pyruva, kinase, Rabies Virus, RAC-1 ant Rac-2, RAGE (receptor for AGE). RANTES, RDX, RccA, Receptor for advanced glycation end products (RAGE), Red Blood cells, Regulatory subunit, RELM alpha and Beta (resistin like molecules), Renin, Rennin, Replication Protein A (RPA p32 anti p70), Resistin, Respiratory syncytial virus (RSV), Retinoblastoma (Rb), phospho-specific RB (ser780), Ribonuclease A, RNA Polymera, Arna3. RNP (70KdaU1), A Protein, B Protein, RO (RO52, Ro60), Rotavirus group specific antigen, Rubella virus structural glycoprotein El, Ryanodine Receptor, S-100 Protein, saccharomyces cerevisiae, Salmonella O-antigens, Salmonel, typhimurium, Sarcosine Oxidase, SDF-1 Alpha and SDF-1 Beta, secretin, Selenoprotein P, Serotonin, Serotonin Receptor. Serotonin Transporter, Sex Hormone Binding Globulin (SHBG), SFRP5 (secreted frizzled-related protein 5), SP21 and SF9, SIV gp120, SIV p28, Smooth muscle actin, Somatostatin Staphylococcus aureus, Staphylococcus aureus enterotoxin, STAT1, Stat2, Stat, Stat4, Stat5 Stat6, Stem Cell Factor (SCF) and SCFR/C-kit, Streptavidin, Streptococcus B, Stromal Cell Derived Factor-1 (SDF-1 alpha and bcta), Substance P, Sufentanil AB, Superoxide Dismutase, Surfactant Associated Proteins (A,B,C,D), Symplekin, Synapsin 1, Synapsin lla. Synaptophysin, Synaptopodin (Podocyte Protein), Syndecan 1, Synphilin-1, Synuclein (alpha), SV40 Large T antigen and small T antigen, Talin, TARC, TAU, Taurine transporter, Tenascin, Testosterone, TGF-alpha, TGF-beta, TGF beta receptor (Endoglin), THC, Thomsen Friedenreich Antigen (TF), TRY-1 25 kd Brain (CDw90), Thymocytes, Thrombin and Thrombin Receptor, Thyroglobulin (24G/5E6 and 24Tg/5F9), Thyroid Binding Globulin Thyroid Hormone Receptors, Thyroid Peroxidase, Thyroid Stimulating Hormone (TSH), Tyrosine Hydroxylase, Thyrotropin Releasing Hormone (TRH). Thyroxine (T4), Tle-1 and Tle-2, TIMP-1, TIMP-2, TIMP-3 (Tissue Inhibitors, metalloproteinase). Titin, TNF receptor associated factors 1 and 2, TNF Receptor, TNF receptor II. TNF-Alpha, TNF-Alpha, TNF-beta, Toxoplasma gondii p30 antigen, TPO (thrombopoietin), TRAF, TraP2,Traf3,TRAF4,TRAF5, TRAF6, Transferrin, Transferrin Receptor, Transforming Growth Factor A, Transformi, Growth Factor Beta, Transportin, Trepone, pallidium, Triiodothyronine (T3), Trinitrotoluene (TNT), TRK A, TRK B, TRK C, Tropon, (cardiac), Troponin I, Troponin T, trypsin, trypsin inhibitor, trypsinogen, TSH, TUB Gene, Tubulin alpha and beta, Tubulin beta specific, Tumor Marker related Antibodies, Tumor Necrosis Factor Alpha Tyrosinase, Tweak, (caspase-4), Ubiquitin, Ubiquitin-L1, Uncoupling Proteins (UCP1, UCP2, UCP3, UCP 4 and UCP5), Urease, Uricase, Urocortin, Uroplakin, Vasopressin, Vasopressin Receptor, VEGF, Vesicular acetycholine transport, (VACht ), Vesicular monoamine transporter (VMAT2), Villin, Vimentin, Vinculin, VIP (Vasoactive Intestinal Peptide), Vitamin B12, Vitamin B12, Vitamin D metabolites, Vitamin D3 Receptor, Von Willebrand Factor, VSV-G Epitope Tag, Wilm's tumor Protein X, Oxida, Yeast, hexokinase, SOD, cytochrome oxidase, carboxypeptidase, and Yersinia eterocolotica.

The list given in the previous paragraph gives examples of compounds that make appropriate analytes for the instant invention. Indeed, a number of different assays—competive, sandwich, ELISA, gel and thin layer, hybridization, etc.—may be performed with the materials of the present invention. As noted above, these materials may have cenlain advantages in terms of reducing or eliminating operations such as washing and aspiration (since the target is maintainedl separate from the media (i.e., within the nanostructured liquid phase or liquid crystalline phase particle or material)); and shipping and handling (again because of the protection of the target by the nanostructured liquid phase or liquid crystalline phase particle or material). The assays may be employed for standard hematology, urology, chemistry screening. Examples of particularly appropriate chemicals which might be tested by assay in hematology, urology and chemistry screening include acetone, acid phosphatase, ACTH, albumin, alkaline phosphatase, ammonia, amylase, vitamins (e.g. B12), bilirubin, calcium cholesterol, cortisol, creatinine, estradiol, ferritin, folic acid, glucose, growth hormone, hemoglobin, hepatitis a, hepatitis b, hepatitis c, HIV, immunoglobulins (IgA, IgE, IgG, IgM), insulin, lipase, luteinizing hormone, lactic acid, myoglobin, presence of elements (e.g., calcium, potassium, oxygen, iron, phosphorous, sodium etc.), progesterone, prolactin, prostate specific antigen, rheumatoid factor, rubella, testosterone, tropinin, uric acid, triglyserides, aldosterone, amylase, Bence Jones Protein, catecholamines, urea, leukocyte antibody, acanthamoeba, chlamydia, clostridium, cytomegalovirus, influenz, phenumocystis Carinnii, rotavirus antigen, RSV, varicella zoster, neisseria gonorrhea, toxoplasma antibody, pinworm, Factors II, V, VII, VIII, IX, X, XI, XII, anrd XIII, lupus, anticardiolipin antibody, antithrombin, protein c, protein S, anti beta 2 glycoprotein, Von Willebrand Factor, etc. Other examples include viral nucleic acids, nucleic acids from other sources where DNA technology has been or could be applied, viral coat proteins (or other proteins in the virus), bacterial adhesins. etc.

Assays might also be performed for therapeutic drug monitoring and toxicology including monitoring of acetaminophen, amidarone, cyclosporin, digoxin, dilantin, FK 506. gentamicin, lidocaine, lithium, methotrexate, norpace, phenobartitol, procaine, quinidine, salicylate, tegretol, theophylline, thiocyanate, tobramycin, valproic acid, and vancomycin. Illicit drugs could also be important analytes in the context of this invention, in particular cocaine, heroin and other opiates. PCP, marijuana, amphetamines, barbituates, LSD and other indole hallucinogens, mescaline, ecstacy, etc.

The invention can be used in other chemical segregation or separation applications where no analysis is performed. For example, hazardous waste clean up may be performed using the chemical segregation or separation devices of this invention where a hazardous chemical (toxin, radiochemical, etc.) is to be removed from a sample or medium. For example, effluent from a industrial discharge may have selected chemicals separated at the discharge outlet or retrieved from water nun off in a creek or riverbed using the chemical separation or segregation devices of the present invention.

In addition to the in vitro applications discussed above, the invention may also be used ex vivo. In an ex vivo application, such as a blood transfusion, chemicals of interest could be separated and, if desired, analyzed, from the blood being transfused.

The invention may also be used for targeted drug delivery in vivo in humans and animals. For example, a compound to be delivered, e.g., an enzyme, medicament, agonist, antagonist, radiotoxin or chemical toxin, nutrient, or the like, can be delivered by administering to a patient a chemical segregating or separating device according to this invention with a target therein having a displacable chemical to be delivered. Administration can be by any suitable means including intraperitoneal, intravenous, subcutaneous, intramuscular, oral, buccal, etc. The chemical to be delivered is protected from degradation in the body by the porous nanostructured liquid or liquid crystalline particle or material. When an agent in the body diffuses into the porous nanostructured liquid or liquid crystalline particle or material, the compound is released and delivered to the patient by diffusion. As one example, the chemical to be delivered might be a seritonin agonist. Upon being displaced by an antagonist, the agonist would be selectively delivered to the patient. Another example would be the targeted delivery of a killing agent to a tumor cell, e.g., P53, methotrexate. Once in the appropriate location, the killing agent would be released to kill the tumor cell.

Polymerized liguid crystals. In the case where the liquid crystalline phase is in the form of unpolymerized particles, the target molecules and associated ligands will be diffusing within the structure, and may at certain moments in time be located close enough to the outer surface of a particle that they can, in principle at least, interact with marker molecules that are by design substantially outside the particles. If this interaction is substained enough that it allows enzymatic (or other, depending on the nature of the detection system in the instant invention) reaction with the marker, then this can lead to a background, i.e., to a signal that is not due to the presence of analyle. While this noise level could be corrected for, the net result would be either a loss of sensitivity or a truncation of the dynamic range.

If one assigns a distance d to the thickness of the layer just within the particle surface (viewed as a precise mathematical surface) within which the enzyime does have access to the marker molecule, and if the particle diameter is D, then the fraction f of the particle volume that lies in this region is f=1-(1-2/D)³. A rough estimate For d might be on the order of 4 nm, the thickness of a typical lipid bilayer in a reversed cubic phase, also roughly equal to the effective diameter of a protein such as peroxidase or phosphatase. Thus, if a particle had a diameter of, say, 200 nm, then this estimate would give f=0.115.

However, this formnula probably greatly overestimates the effect, for several reasons. First, it is well known that for high-MW polymers, such as those used as markers in many of the embodiments of the instant invention, in the presence of particles, a so-called “depletion layer” exists just outside the surface of the particles from which the polymer is substantially excluded, due to steric interactions. Since this depiction layer is particularly pronouned when the MW of the polymer is very high, and a design criterion of the marker molecule in many embodiments of the instant invention is that the MW be very high so as to be sterically excluded from the pores of the particles, it should be expected that this depletion layer constitutes a significant impediment against enzyme-marker contact near the particle surface. Second, due to the fact that most proteins, including even soluble enzymes, have favorable interactions with lipid membranes, it can be expected that proteins will often partition preferentially into the interior of a liquid crystalline phase. The presence of a hydrophobic portion of the protein, such as an alpha-helix in the appropriate pH range or an acyl chain anchor, can be sufficient to induce such partitioning. This is particularly true in the case of the instant invention, since the target protein is purposefully chosen so as to have strong favorable interactions with hydrophobic domains of the liquid crystal. And third, one can reasonably assume that the ability of the enzyme to react withhl the marker will be hindered—once again, due to steric interactions—when the enzmne is still bound to the target, that is, before it has been displaced by competitive binding of analyte, whether it is at the particle surface or not. The combined effect of some or all three of these steric factors will be to reduce the background due to the presence of enzyme at the particle surface. Furthermore, the design of an optinized system can involve minimizing this background contribution through as many means as possible. For example, an anionically-charged marker molecule could he used together with an anionic bilayer component in the liquid crystal, to yield an additional marker-particle repulsion, this time ionic in nature, to further limit contact between the marker and components at the particle surface.

Another embodiment of the invention can be used to greatly reduce, and perhaps virtually eliminate, this background effect. Particles, preferably nanoparticles with diameters less than about 200 nm, and moire preferably less than about 100 nm, with target molecules attached at their surlaces, could be coated with cubic phase or other porous liquid crystal. Provided that the coating is complete—which would not be difficult to achieve, since the surface energy of a cubic phase is much less than that of many solids commonly used to attached proteins, such as silica—then there would be essentially no marker molecules (nor enzyme) at the particle surface. Diffusion of the analyte and displaced enzyme would still occur through the pores of the liquid crystal, and these pores would exclude marker from contact with undisplaced enzyme. One means to accomplish this coating would be to covalently attach the target molecules to a dispersion of the nanoparticles using any of a wide range of standard chemistries for protein conjugation, then mix the particles into a bulk cubic phase with the removal of most of the water (leaving only that water which is necessary for the formation of the cubic phase). The cubic phase would then be dispersed according to techniques as described elsewhere herein, or dispersed as coated particles. If dispersed as coated particles, then the coating would have to be removed (usually dissolved) before any assay were performed; however, the presence of the coating could provide a superior format in which to store the system so as to achieve maximum shelf life.

Polymerization of the liquid crystal, as for example by techniques described in U.S. Pat. Nos. 5,244,799 or 5,238,613, each of which are herein incorporated by reference, could provide advantages as to stability (especially as regards shelf-life) and to control of microstructure and diffusion. Changes in phase, or in microstructural dimensions even within the same region of the phase diagram, are known to occur with the addition of relatively small amounts of proteins and other compounds and polymerization can provide a means by which to stabilize the phase and the poresize against such effects. Furthermore, diffusion of membrane-bound components, in particular target molecules such as receptors and antibodies, can be severely restricted by polymerization of bilayer components as described in U.S. Pat. No. 5,244,799, and this can provide a means to lower the background. This is particularly true in the case of the sandwich assays performed as described in the instant invention, since it would severely limit the ability of two target antibodies in the cubic phase bilayer to form a sandwich with the analyte; rather, the much greater mobility of the soluble marker would strongly favor the desired sandwich containing both the soluble marker and the cubic phase-bound target. In the case of polymerization of a bilayer component of the cubic phase, polymerization could be performed either after dispersing the cubic phase (in which case coating of the particles would provide a means to avoid polymerizing two or more particles together), or before dispersing, in which the process of dispersing the cubic phase would require somewhat higher energy input to overcome the effect of the polymerization of the bilayer.

Polymerization according to U.S. Pat. No. 5,238,613 provides a means by which to embed a marker polymer in the cubic phase, in particular in the aqueous channels of the cubic phase. As above, the polymerization in such a case could be done after dispersing the cubic phase into coated particles, so that the coating would prevent linking between particles or leakage of the monomer into the exterior phase with subsequent polymerization. Alternatively, in the case where the enzyme were bound inside the cubic phase and the marker outside, the polymerization of an aqueous mononmer such as acrylamide were performed in such a way that the polymer became crosslinked (i.e., a hydrogel), then this would retard the diffusion of enzyme just as in the case of electrophoresis. This could be used to slow the enzyme's escape from the cubic phase, in cases where it would be advantageous to slow the response time to obtain more quantitative information (i.e., if the kinetics in the absence of the crosslinked polymer were too fast for good quantitation).

### EXAMPLE 1

Experiment 1. A dispersion of microparticles containing acetylcholine receptor protein was first prepared. An amount of 0.470 gratns of phosphatidylcholine-rich soy lecithin (Epikuron 200, from Lucas-Meyer) was mixed with 0.183 grams of sorbitan trielacostearate, and 0.359 grams or water. To this was added 0.112 grams of potassium carbonate. This was centrifiged for several hours and the excess aqueous phase removed. In the receptor preparation (obtained from Dr. Mark McNamee of U C Davis), 50 micrograms of receptor protein was contained in 50 microliters of lipid, most or which was, dioleoylphosphatidylcholine (DOPC). This amount of preparation was added to the cubic phase-potassium carbonate mixture, and the entire mixture stirred gently but long enough to ensure good mixing, as checked by the absence of birefringence. An upper solution was prepared by adding 0.328 grams of magnesium sulfate, 0.324 grams of Pluronic F-68, and 0.0722 grains of cetylpyridinium bromide to 20.02 grams of water. Five grams of the upper solution were overlaid onto the test tube containing the receptor-loaded cubic phase. and the test tube scaled, shaken, and sonicated for 2 hours. This resulted in a dispersion of receptor-containing microparticles, a substantial fraction of which were in the side range of a 0.5 to 1 micron.

Although a gelation was carried out in this instance, in order to render the dispersion more easily handled during an assay that was more akin in some respects to a solid-phase assay, the results clearly indicate that the receptor protein remained active anal accessible in the cubic phase microparticle dispersion. For the gelation, acrylamide (0.296 grams), methylene-bis-acrylamide (0.024 grams, as crosslinker), ammonium persulfate (0.005 grams, as initiator), and tetramethylethylene diamine (TMED, 0.019 grams, as co-initiator) were added to the dispersion, resulting in polymerization of the acrylamide into a crosslinked hydrogel in less than 30 minutes. A thin slice of the hlydrogel was examined under a microscope, and a high concentration of micropanicles was seen, just as with the original dispersion.

Thus, using ¹²⁵I-labeled bungarotoxin as the ligand, an assay of receptor binding was performed using the cubic phase microparticle-immobilized acetylcholine receptor system. A standard assay for binding was performed wherein the labeled bungarotoxin is incubated with the receptor-containing preparation for one hour, after which the entire suspension is passed over a DE-AE filter, which retains the beads but allows free toxin to pass through in the filtrate. The filter, and any deposited beads, are then counted in a scintillation counter to quantify the amount of ¹²⁵I-labelled bungarotoxin present.

In the case of the hydrogel beads prepared in this experiment, the beads were first washed, in order to remove the particle coating on the embedded cubic phase microparticles, by dissolution. In the case of the magnesiumn carbonate hydroxide coated particles, a final wash with salt water was necessary, in order to displace any bound magnesium ions from the receptor.

The results showed that the cubic phase microparticle-immobilized acetylcholine receptor system exhibited binding of the bungarotoxin at approxiihntely 70% of the level measured with the standard receptor preparation, demonstrating the retention of protein binding properties throughout not only the immobilization procedure but also the transcontinental mailings and several months of storage time. Indeed, the untouched lipid-receptor preparation, after some three months in a freezer and two transcontinental shippings, was found to have retained only 48% of its original activity: the receptor binding was measured to be 2068 picomoles per milligram of preparation, in contrast with 4335 for freshly prepared receptor. Taking this into account, the conclusion is that within the limits of the accuracy, there was no loss of binding activity associated with the cubic phase—hydrogel itnnobilization procedure.

### EXAMPLE 2

Experimnent 2. The dispersion prepared it Experiement 1, without the gelution step, is first treated with nitrophenylphosphate-labelled bungarotoxin, by adding it to thc exterior phase of the liquid dispersion at approximately an equimiolar amount to the receptor protein. After this is equilibrated for several hours, alkaline phosphate is added and the pH is adjusted to 8.5 with a standard buffer for alkaline phosphatase action. This system is thus able to dtect any analyte, such as acetylcholine, that can bind to the AChR in a manner that is competitive with the labeled bungarotoxin. In particular, the absorbance at 285 nm is seen to increase as acetylcholine is added to the dispersion, indicating the action of alkaline pbosphatase on the nitrophenylphosphate group of the displaced toxin.

### EXAMPLE 3

Experimental results—A cubic phase was first. prepared by mixing 0.752 grams of Pluronic P123 (an insoluble surfactant), 0.705 grams of linalool, and 0.703 grams of water. An amount of 1.005 grams of this cubic phase was put in a glass flask together with 0.054 grams of the rhamnolipid surfactant JBR-99 (Jeneil Biosurfactant, Inc.) and 35 ml of pH 4.5 acetate buffer containing 4 mM MnCl₂ and 4 mM CaCl₂. The flask was then sonicated to disperse the cubic phase. Following this, the dispersion was microfluidized in a model 110S Microfluidizer (Microfluidics, Inc.) to a particle size that was fine enough where the absorbance measured on an Ultrospec 3000 UV-V is spectrometer, at a wavelength of 620 nm, was about 0.2 absorbance units.

The following reagents were then added to 2 ml of the cubic phase dispersion:

Anti Concanavalin A, Vector AS-2004. Lot 0321, 1 mg/ml stock solution prepared; working solution prepared by diluting 1:10 to 0.1 mg/ml: 51 microliters added.
Concanavalin A. Sigma C-5275, Lot 60K8934 prepared as 1 mg/ml stock solution; working solution prepared by diluting 1:10 to 0.1 mg/ml: 16 microliters added.
Biotinylated mannotriose, V-labs, NGB1336, prepare a 1 mg/ml stock solution, working solution prepared by diluting 1:100 to 0.01 mg/ml: 20 microliters added.
HRP/Avidin; 0.28 mg/ml stock solution: 90 microliters added.

Fifteen minutes were allowed for diffusion and equilibration after the addition of the antibody and Con A solutions. Another fifteen minutes were allowed after the addition of the biotinylated mannatriose and HRP/avidin. This is an important step, because if insufficient time is allowed for the HRP to bind inside the cubic phase microparticles, this would be seen immediately in the subsequent assay, since in the presence of HRP outside the particles, enzymatic action occurs before the displacement step. In practicing the invention, the target and cubic phase should be exposed for a sufficient time or under suitable conditions so as to allow an equilibrium to be achieved.

The Detection System was then added. To 10 drops of a Dextran Blue solution, at 3.9 mg/ml water, were added 6 drops of fast red TR salt, 2.4 mg/ml, 1 drop of 3% H₂O, and 800 ul 50 mM sodiuin acctate pH 4.5 containing 4 mM MnCl₂ and 4 mM CaCl₂. This solution has been found to show disappearance of absorbance at 620 nm upon addition of HRP, or the entire antibody-Con A-biotinylated mannatriose-avidin/HRP. At the end of all these additions, the total volume in the cuvette was 3.0 ml.

After the addition of the Dextran Blue-based Detection System, absorbance readings at 620 nm were monitored continuously. After the readings stabilized at 0.40 absorbance units, 500 microliters of Displacement Solution were added. This solution was composed of saturated alpha methylmannoside in 50 mM sodium acetate pH 4.5 containing 4 mM MnCl, and 4 mM CaCl₂.

Upon addition of this alpha methylmannoside—the analyte—the absorbance dropped from 0.40 to 0.26 absorbance units. This decrease, 35%, is far greater than the 14% that one would expect based on the dilution from 3.0 to 3.5 ml volume, and was reproducible, as seen in several repetitions. The majority of the decrease in absorbance was due to the enzymatic action of displaced HRP on the Dextran Blue.

These results demonstrates a homogeneous assay of the instant invention for the detection of saccharides. Other analytes would be readily detectable under the same principles, and further, the results demonstrate that the segregating aspect of the invention separate and apart from an assay system would perform similarly.

### EXAMPLE 4

This Example illustrates the production of particles that have a water-soluble solid coating, so that dispersions of microparticles of nanostructured liquid crystalline phases can be easily and conveniently produced simply by placing the material in water or buffer. The solid coating can protect the liquid crystal and components therein during later production stages and, most importantly, during product storage time.

A cubic phase containing solubilized mnethyl red was first prepared by mixing 2.118 grams of Arlatone G, 0.904 grams of water, 1.064 grams of oil of ginger, and 0.012 grams ol methyl red, and stirring thoroughly. A trehalose solution was prepared by dissolving 2.00 grams of trehalose in 10.005 grams of water. Then 1.002 grams of the cubic phase were dispersed in the trehalose solution by a combination of shaking and mild sonication. This dispersion was then freeze-dried in a lyophilizer. Trehalose solutions are known to yield amorphous solid on freeze-drying. The resulting material flowed freely, and gave no hint of the greasy, sticky feel and behavior that characterizes the uncoated cubic phase. There was no second phase present, as the material was homogeneous to the eye, and had a strong, uniform, red-orange color. A large particle of the material could be speared with the point of a push-pin and held firmly in place without deforming under gravity; an uncoated cubic phase would not have been possible to spear in this fashion.

In the phase-contrast optical microscope, thin portions of this material were readily seen to contain a fine-scale structure, which is consistent with the presence of cubic phase microparticles (submicron to 5 microns in size) within the trehalose solid matrix. The material was brittle and could therefore be crushed into small particles with ease. Upon mixing the material into water at a 1:10 ratio, a dispersion was immediately obtained which was indistinguishable in the optical microscope from dispersions of this cubic phase in water.

Since methyl red is a water-insoluble compound, it will partition strongly into the cubic phase in the application of particles such as these in an assay system.

While the invention has been described in terms of its preferred embodiments, those skilled in the art will recognize that the invention can he practiced with modification within the spirit and scope of the appended claims.

1-88. (canceled)

89. A method for performing an assay, comprising the steps of:
adding to a medium which contains or is suspected to contain at least one chemical of interest and a marker which undergoes a detectable change, one or more a porous nanostructured liquid or liquid crystalline particles or materials selected from the group consisting of reversed bicontinuous cubic phase, reversed hexagonal phase, L3 phase, normal bicontinuous cubic phase, and normal hexagonal phase, or a dehydrated variant thereof, wherein said porous nanostructured liquid or liquid crystalline particles or materials that includes a target which binds said at least one chemical of interest with specificity located inside an external surface of said porous nanostructured liquid or liquid crystalline particle or material, where said target is only accessible by said at least one chemical of interest by diffusing into said porous nanostructured liquid or liquid crystalline particle or material; and
detecting, without washing said porous nanostructured liquid or liquid crystalline particles or materials, when said at least one chemical is bound by said target using said marker.

90. The method of claim 89 wherein said medium is a liquid.

91. The method of claim 89 wherein said medium comprises blood.

92. The method of claim 89 wherein said medium comprises urine.

93-97. (canceled)

98. The method of claim 89 wherein said marker is characterized by being prevented from diffusing into said porous nanostructured liquid or liquid crystalline particle or material.

99. The method of claim 89 wherein said marker is selected from the group consisting of chemiluminescent, phosphorescent, fluorescent, calorimetric, absorbance changing, and conductance changing substrates.

100. The method of claim 89 wherein said marker is of a size and chemical constitution that permits diffusion into said porous nanostructured liquid or liquid crystalline particle or material.

101. The method of claim 89 further comprising the step of displacing an enzyme bound to said target with said chemical of interest.

102. The method of claim 101 wherein said enzyme is of a size and chemical constitution that permits diffusion out of said porous nanostructured liquid or liquid crystalline particle or material.

103. The method of claim 101 wherein an enzyme which acts on said marker in said detecting step.

104. (canceled)

105. The method of claim 89 further comprising the step of diffusing a second target into said nanostructured liquid or liquid crystalline particles or materials, said second target binds said at least one chemical of interest with specificity at the same time said target binds said at least one chemical of interest.

106. The method of claim 89 wherein said at least one specific chemical is an analyte selected from the group consisting of hormones, neurotransmitters, peptides, proteins, antibodies, soluble receptors, viruses, nucleic acids, endotoxins, microbial products, sugars, and bioactive compounds.

107. The method of claim 89 wherein said at least on specific chemical is a therapeutic drug.

108. A method of administering a chemical to a patient, comprising:
providing said patient with a one or more porous nanostructured liquid or liquid crystalline particles or materials selected from the group consisting of reversed bicontinuous cubic phase, reversed hexagonal phase, L3 phase, normal bicontinuouis cubic phase, and normal hexagonal phase or a dehydrated variant thereof, wherein said porous nanostructured liquid or liguid crystalline particles or materials include a target which binds at least one chemical with specificity located in said porous nanostructured liquid or liquid crystalline particles or materials, said target being accessible by said at least one chemical by diffusing into said porous nanostructured liquid or liquid crystalline particles or materials, wherein a bioactive agent is bound to said target and is displaceable by said at least one chemical; and
displacing said biologically active agent with said at least one chemical, said biologically active agent being of a size and a chemical constitution which permits diffusion out of said porous nanostructured liquid or liquid crstalline particles or materials.

109. The method of claim 108 wherein said biologically active agent is an enzyme.

110. (canceled)

111. The method of claim 108 wherein said biologically active agent is a medicament.

112. The method of claim 108 wherein said biologically active agent is a reeptor agonist or antagonist.

113. The method of claim 108 wherein said biologically active agent is lethal to surrounding tissues or cells.

114-116. (canceled)

117. A method of performing an assay comprising the steps of:
preparing a dispersion of nanoporous particles and a marker together in a medium,
wherein said nanoporous liquid or liquid crmstalline particles are selected from the group consisting of reversed bicontinuouss cubic phase, reversed hexagonal phase, L3 phase, normal bicontinuous cubic phase, and normal hexatonal phase, or a dehydrated variant thereof, wherein said nanoporous liquid or liquid crystalline particles incorporate within an external surface a target which binds at least one chemical with specificity, said target being accessible by at least one chemical by diffusing into said nanoporous liquid or liguid crvstalline particles, and
wherein said marker undergoes a detectable change when said at least one chemical is bound by said target; and
detecting a change in said marker due to binding of said at least one chemical bound by said target.

118-119. (canceled)
